(19) United States
(12) Patent Application Publication
(10) Pub. No.: US 2006/0287251 A1
(43) Pub. Date: Dec. 21, 2006

(54) Combination therapy for glycaemic control

(76) Inventors: Hans-Ulrich Demuth, Halle/Saale (DE); Konrad Glund, Halle/Saale (DE); Matthias Hoffmann, Halle/Saale (DE)

Correspondence Address: OSI PHARMACEUTICALS, INC., 41 PINELAWN ROAD, MELVILLE, NY, 11747, US

(21) Appl. No.: 10/570,113

(22) PCT Filed: Sep. 2, 2004
(86) PCT No.: PCT/IB04/03082

§ 371 Date: Mar. 1, 2006

Related U.S. Application Data
(60) Provisional application No. 60/499,535, filed on Sep. 2, 2003.

(51) Int. Cl. A61K 38/04; A61K 31/426; A61K 31/401

(52) U.S. Cl. 514/19; 514/365; 514/423

(57) ABSTRACT

The present invention relates to method of treatment, in particular to a method for the treatment of diabetes mellitus, especially non-insulin dependent diabetes mellitus (NIDDM) or Type 2 diabetes and conditions associated with diabetes mellitus the predi,betic state and/or obesity and to compositions for use in s ch method.

### FIELD OF THE INVENTION

This invention relates to a therapy for glycaemic control, in particular to a method for the treatment of diabetes mellitus, especially non-insulin dependent diabetes mellitus (NIDDM) or Type 2 diabetes and conditions associated with diabetes mellitus, the prediabetic state and/or obesity and to compositions for use in such method.

### BACKGROUND ART

Glycaemic control is therapeutically important in the treatment of conditions such as diabetes mellitus and related conditions. Clinical diabetes may be divided into four general subclasses, including (1) type 1 or insulin-dependent diabetes mellitus (IDDM) (caused by beta cell destruction and characterized by absolute insulin deficiency), (2) type 2 or non-insulin-dependent diabetes (NIDDM) (characterized by insulin resistance and relative insulin deficiency, (3) other specific types of diabetes (associated with various identifiable clinical conditions or syndromes such as genetic defects of β-cell function e.g. maturity-onset diabetes of the young [MODY] types 1-3 and point mutations in mitochondrial DNA), and (4) gestational diabetes mellitus.

Type 2 diabetes is by far the most common form of the disease, is found in over 90% of the diabetic patient population. These patients retain a significant level of endogenous insulin secretory capacity. However, insulin levels are low relative to the magnitude of insulin resistance and ambient glucose levels. Type 2 patients are not dependent on insulin for immediate survival and ketosis rarely develops, except under conditions of great physical stress. Nevertheless, these patients may require insulin therapy to control hyperlgycemia. Type 2 diabetes typically appears after the age of 40 years, has a high rate of genetic penetrance unrelated to specific immune response (HLA) genes, and is associated with obesity.

In addition to these clinical categories, further conditions, namely impaired glucose tolerance and impaired fasting glucose, refer to a metabolic state intermediate between normal glucose homeostasis and overt diabetes (under fed and fasting conditions, respectively). These conditions significantly increase the later risk of diabetes mellitus and may in some instances be part of its natural history.

A further related condition is Impaired Glucose Metabolism (IGM) which is defined by blood glucose levels that are above the normal range but are not high enough to meet the diagnostic criteria for type 2 diabetes mellitus. The incidence of IGM varies from country to country, but usually occurs 2-3 time more frequently than overt diabetes. Among subjects with IGM, about 58% have Impaired Glucose tolerance (IGT), another 29% have impaired fasting glucose (IFG), and 13% have both abnormalities (IFG/IGT).

Many of the available treatments for type 2 diabetes, which have not changed substantially in many years, have recognized limitations for example they may have unwanted side effects, low efficacy or suffer from efficacy loss over time during chronic treatment.

Increasing the plasma level of insulin by administration of sulfonylureas (e.g. tolbutamide and glipizide) or meglitinide, which stimulate the pancreatic (β-cells to secrete more insulin, and/or by injection of insulin when sulfonylureas or meglitinide become ineffective, can result in insulin concentrations high enough to stimulate the very insulin-resistant tissues. However, dangerously low levels of plasma glucose can result from administration of insulin or insulin secretagogues (sulfonylureas or meglitinide), and an increased level of insulin resistance due to the even higher plasma insulin levels can occur. Alpha glucosidase inhibitor antihyperglycaemic agents (or alpha glucosidase inhibitors) and biguanide antihyperglycaemic agents (or biguanides) which increase insulin sensitivity resulting in some correction of hyperglycemia, are commonly used in the treatment of type 2 diabetes. Acarbose, voglibose, emiglitate and miglitol are examples of alpha glucosidase inhibitors. 1,1-Dimethylbiguanidine (or metformin) and phenformin are particular examples of biguanides, metformin has fewer side effects than phenformin.

The glitazones (i.e. 5-benzylthiazolidine-2,4-diones) are a more recently described class of compounds with potential for ameliorating many symptoms of type 2 diabetes. These agents substantially increase insulin sensitivity in muscle, liver and adipose tissue in several animal models of type 2 diabetes resulting in partial or complete correction of the elevated plasma levels of glucose without occurrence of hypoglycemia. The glitazones that are currently marketed are agonists of the peroxisome proliferator activated receptor (PPAR), primarily the PPAR-gamma subtype. PPAR-gamma agonism is generally believed to be responsible for the improved insulin sensititization that is observed with the glitazones. Newer PPAR agonists that are being tested for treatment of Type 2 diabetes are agonists of the alpha, gamma or delta subtype, or a combination of these, and in many cases are chemically different from the glitazones. Side effects (e.g. liver toxicity) have occurred with some of the glitazones, such as troglitazone.

New approaches to the treatment of type 2 diabetes that have been recently introduced or are still under development include treatment with alpha-glucosidase inhibitors (e.g. acarbose) and protein tyrosine phosphatase-1B(PTP-1B) inhibitors.

Insulin secretagogues are compounds that promote increased secretion of insulin by the pancreatic beta cells. The sulphonylureas are well known examples of insulin secretagogues. The sulphonylureas act as hypoglycaemic agents and are used in the treatment of Type 2 diabetes. Examples of sulphonylureas include glibenclamide (or glyburide), glipizide, gliclazide, glimepiride, tolazamide and tolbutamide.

European Patent Application 0306228 discloses certain thiazolidinedione derivatives disclosed as having antihyperglycaemic and hypolipidaemic activity, for example 5-[4-[2-(N-methyl-N-(2-pyridyl) amino)ethoxy]benzyl]thiazolidine-2,4-dione (rosiglitazone). WO 094/05659 discloses certain salts of this compound including the maleate salt thereof. 5-[4-[2-(N-Methyl-N-(2-pyridyl) amino)ethoxy]benzyl]thiazolidine-2,4-dione is an example of a class of antihyperglycaemic agents known as ‘insulin sensitisers’. In particular this compound is a thiazolidinedione insulin sensitiser. 5-[4-[2-(N-Methyl-N-(2-pyridyl)amino)ethoxy]-benzyl]thiazolidine-2,4-dione is also a peroxisome proliferator-activated receptor (PPARy) agonist insulin sensitiser.

European Patent Applications 0008203, 0139421, 0032128, 0428312, 0489663, 0155845, 0257781, 0208420, 0177353, 0319189, 0332331, 0332332, 0528734 and 0508740; International Patent Applications WO 92/18501, WO 93/02079 and WO 93/22445 and U.S. Pat. Nos. 5,104,888 and 5,478,852, also disclose certain thiazolidinedione insulin sensitisers.

Another series of compounds generally recognised as having insulin sensitiser activity are those typified by the compounds disclosed in International Patent Applications WO 93/21166 and WO 94/01420. These compounds are herein referred to as “acyclic insulin sensitisers”. Other examples of acyclic insulin sensitisers are disclosed in U.S. Pat. No. 5,232,945 and International Patent Applications WO 92/03425 and WO 91/19702. Examples of other insulin sensitisers are disclosed in European Patent Application 0533933, Japanese Patent Application 05271204 and U.S. Pat. No. 5,264,451.

Dipeptidyl peptidase IV (DP IV) is a serine protease which cleaves N-terminal dipeptides from a peptide chain containing, preferably, a proline residue in the penultimate position. Although the biological role of DP IV in mammalian systems has not been completely established, it is believed to play an important role in neuropeptide metabolism, T-cell activation, attachment of cancer cells to the endothelium and the entry of HIV into lymphoid cells.

Likewise, it has been discovered that DP IV is responsible for inactivating glucagon-like peptide-1 (GLP-1) and glucose-dependent insulinotropic peptide also known as gastric-inhibitory peptide (GIP). Since GLP-1 is a major stimulator of pancreatic insulin secretion and has direct beneficial effects on glucose disposal, in WO 97/40832 and U.S. Pat. No. 6,303,661 inhibition of DP IV and DP IV-like enzyme activity was shown to represent an attractive approach e.g. for treating non-insulin-dependent diabetes mellitus (NIDDM).

It is known that DP IV inhibitors may be useful for the treatment of impaired glucose tolerance and diabetes mellitus (International Patent Application WO 99/61431, Pederson R. A. et al, Diabetes. 1998 August; 47(8):1253-8 and Pauly R. P. et al, Metabolism 1999 March; 48(3):385-9).

WO 99/61431 discloses DP IV inhibitors comprising an amino acid residue and a thiazolidine or pyrrolidine group, and salts thereof, especially L-threo-isoleucyl thiazolidine, L-allo -isoleucyl thiazolidine, L-threo-isoleucyl pyrrolidine, L-allo-isoleucyl thiazolidine, L-allo-isoleucyl pyrrolidine, and pharmaceutically acceptable salts thereof. WO 03/072556 discloses the DP IV inhibitors glutaminyl thiazolidine and glutaminyl pyrrolidine and pharmaceutically acceptable salts thereof.

It is the object of the present invention to provide new therapies for glycaemic control for example in the treatment of diabetes mellitus, especially non-insulin dependent diabetes (NIDDM) or Type 2 diabetes, conditions associated with diabetes mellitus, the pre-diabetic state and/or obesity, which may exhibit greater efficiency and/or safety. In particular the present invention provides the use of combinations of the DP IV-inhibitors glutaminyl thiazolidine and glutaminyl pyrrolidine and other antidiabetic agents for glycaemic control, for example in the treatment of diabetes mellitus, especially non-insulin dependent diabetes (NIDDM) or Type 2 diabetes, conditions associated with diabetes mellitus, the pre-diabetic state and/or obesity.

### SUMMARY OF THE INVENTION

The present invention provides a method for glycaemic control in a mammal, such as a human, which method comprises administering an effective amount of glutaminyl thiazolidine or glutaminyl pyrrolidine, or a pharmaceutically acceptable salt thereof, and another antidiabetic agent, to a mammal in need thereof.

The invention also provides the use of glutaminyl thiazolidine or glutaminyl pyrrolidine, or a pharmaceutically acceptable salt thereof, and another antidiabetic agent for glycaemic control.

The invention also provides the use of glutaminyl thiazolidine or glutaminyl pyrrolidine, or a pharmaceutically acceptable salt thereof, in the manufacture of a medicament for use in combination with another antidiabetic agent, for glycaemic control.

Glutaminyl thiazolidine and glutaminyl pyrrolidine have the following structure: 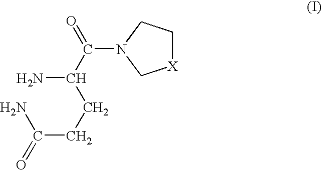
wherein for glutaminyl thiazolidine X=S and for glutaminyl pyrrolidine X=CH₂.

These compounds are hereinafter referred to as compounds of formula (I).

The combinations described above are of particular use for the treatment of diabetes mellitus, especially Type 2 diabetes, and conditions associated with diabetes mellitus, the prediabetic state and/or obesity. In particular the treatment of Type 2 diabetes.

### BRIEF DESCRIPTION OF THE FIGURES

FIG. 1 plots the blood glucose level over time for placebo, and three administered levels of glutaminyl pyrrolidine.

FIG. 2 plots the blood glucose level over time for placebo, and three administered levels of glutaminyl thiazolidine.

FIG. 3 is a chemical drawing of glutaminyl thiazolidine.

FIG. 4 is a chemical drawing of glutarninyl pyrrolidine.

FIG. 5 is a plot of the counts per second over time of glutaminyl thiazolidine and pyroglutamic acid thiazolidine.

FIG. 6 shows the glucose AUC for various administered compositions.

FIG. 7 shows the glucose AUC for various administered compositions.

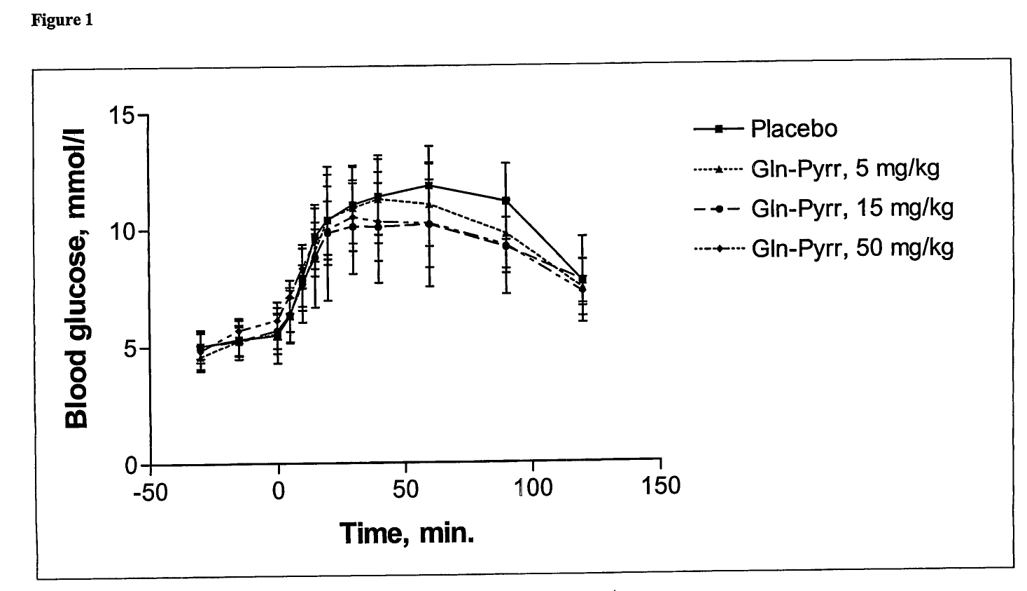

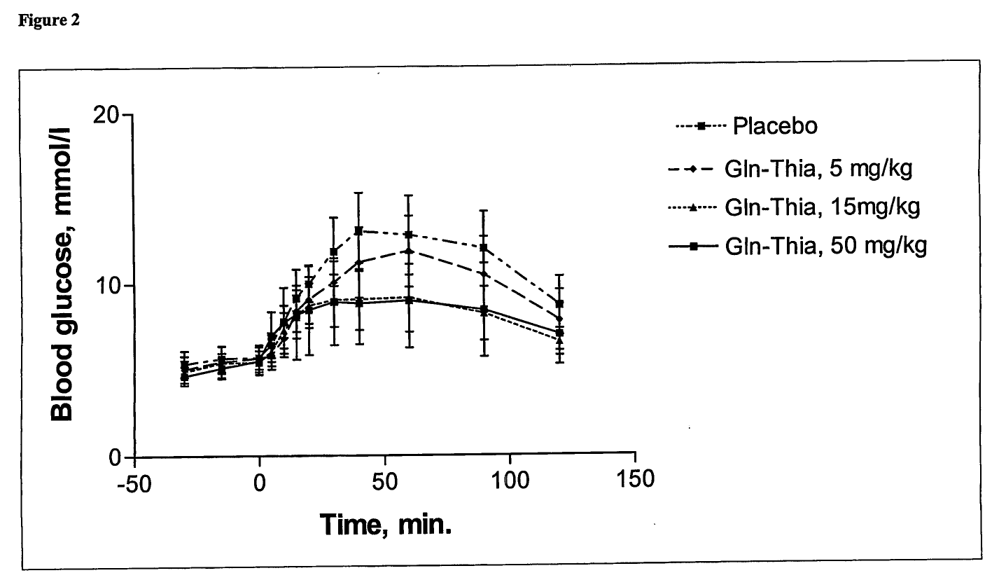

### DETAILED DESCRIPTION OF THE INVENTION

The present invention provides a method for glycaemic control in a mammal, such as a human, which method comprises administering an effective amount of glutaminyl thiazolidine or glutaminyl pyrrolidine, or a pharmaceutically acceptable salt thereof, and another antidiabetic agent, to a mammal in need thereof.

The combinations are of particular use for the treatment of diabetes mellitus, especially Type 2 diabetes, and conditions associated with diabetes mellitus, the prediabetic state and/or obesity. In particular the treatment of Type 2 diabetes.

Such combinations provide a particularly beneficial effect on glycaemnic control and preferably provide improved blood glucose regulation without introducing unacceptable side-effects

The present invention also provides a method for the treatment of diabetes mellitus, especially Type 2 diabetes, and conditions associated with diabetes mellitus, the prediabetic state and/or obesity, in particular the treatment of Type 2 diabetes, in a mammal, such as a human, which method comprises administering an effective amount of glutaminyl thiazolidine or glutaminyl pyrrolidine, or a pharmaceutically acceptable salt thereof, and another antidiabetic agent, to a mammal in need thereof.

The invention also provides the use of glutaminyl thiazolidine or glutaminyl pyrrolidine, or a pharmaceutically acceptable salt thereof, and another antidiabetic agent for the treatment of diabetes mellitus, especially Type 2 diabetes, and conditions associated with diabetes mellitus, the prediabetic state and/or obesity, in particular the treatment of Type 2 diabetes.

The invention also provides the use of glutaminyl thiazolidine or glutaminyl pyrrolidine, or a pharmaceutically acceptable salt thereof, in the manufacture of a medicament for use in combination with another antidiabetic agent, for the treatment of diabetes mellitus, especially Type 2 diabetes, and conditions associated with diabetes mellitus, the prediabetic state and/or obesity, in particular the treatment of Type 2 diabetes.

The compound of formula (I) and the other antidiabetic agent may be co-administered or administered sequentially or separately.

Co-administration includes administration of a formulation which includes both the compound of formula (I), or a pharmaceutically acceptable salt thereof and the other antidiabetic agent, or the essentially simultaneous administration of separate formulations of each agent. Where the pharmacological profiles of the compound of formula (I), or a pharmaceutically acceptable salt thereof, and the other antidiabetic agent allow it, coadministration of the two agents is preferred.

The invention also provides the use of glutaminyl thiazolidine or glutaminyl pyrrolidine, or a pharmaceutically acceptable salt thereof, and another antidiabetic agent, in the manufacture of a medicament for glycaemic control.

The invention also provides the use of glutaminyl thiazolidine or glutaminyl pyrrolidine, or a pharmaceutically acceptable salt thereof, and another antidiabetic agent, in the manufacture of a medicament for the treatment of diabetes mellitus, especially Type 2 diabetes, and conditions associated with diabetes mellitus, the prediabetic state and/or obesity, in particular the treatment of Type 2 diabetes.

The invention also provides a pharmaceutical composition comprising glutaminyl thiazolidine or glutaminyl pyrrolidine, or a pharmaceutically acceptable salt thereof, and another antidiabetic agent, and a pharmaceutically acceptable carrier. The invention also encompasses the use of such compositions in the methods described above.

The present invention includes the use of compounds of formula (I) and pharmaceutically acceptable salts thereof, according to any one of the embodiments of the present invention in combination with:
insulin sensitizers selected from the group consisting of PPAR agonists, biguanides, and protein tyrosin phosphatase-1B (TP-1B) inhibitors;
insulin and insulin mimetics;
sulfonylureas and other insulin secretagogues;
α-glucosidase inhibitors;
glucagon receptor agonists;
GLP-1; GLP-1 mimetics, e.g. NN-2211 (liraglutide from Novo Nordisk), and GLP-1 receptor agonists;
GLP-2; GLP-2 mimetics, e.g. ALX-0600 (teduglutide from NPS Allelix Corp.) and GLP-2 receptor agonists;
exendin-4 and exendin-4 mimetics, e.g. exenatide (AC-2993, synthetic exendin-4 from Amylin/Eli Lilly);
GIP, GIP mimetics, and GIP receptor agonists;
PACAP, PACAP mimetics, and PACAP receptor 3 agonists;
cholesterol lowering agents selected from the group consisting of HMG-CoA reductase inhibitors, sequestrants, nicotinyl alkohol, nicotinic acid and salts thereof, PPARα agonists, PPARα/γ dual agonists, inhibitors of cholesterol absorption, acyl CoA:cholesterol acyltransferase inhibitors, and antioxidants; and
PPARδ agonists;
   and optionally other agents for example:
antiobesity compounds;
an ileal bile acid transporter inhibitor; and
anti-inflammatory agents.

Suitably, the other antidiabetic agent comprises one or more, generally one or two, and especially one, of an alpha glucosidase inhibitor, a biguanide, an insulin secretagogue or an insulin sensitiser.

A further suitable antidiabetic agent is insulin.

A suitable alpha glucosidase inhibitor is acarbose.

Other suitable alpha glucosidase inhibitors are emiglitate and miglitol. A further suitable alpha glucosidase inhibitor is voglibose.

Suitable biguanides include metformin, buformin or phenformin, especially metformin.

Suitable insulin secretagogues include sulphonylureas.

Suitable sulphonylureas include glibenclamide, glipizide, gliclazide, glimepiride, tolazamide and tolbutamide. Further sulphonylureas include acetohexamide, carbutamide, chlorpropamide, glibornuride, gliquidone, glisentide, glisolamide, glisoxepide, glyclopyamide and glycylamide. Also included is the sulphonylurea glipentide.

A further suitable insulin secretagogue is repaglinide. An additional insulin secretagogue is nateglinide.

Insulin sensitisers include PPARy agonist insulin sensitisers including the compounds disclosed in WO 97/31907 and especially 2-(1-carboxy-2-{4-{2-(5-methyl-2-phenyl-oxazol-4-yl) ethoxy]phenylethylamino)benzoic acid methyl ester and 2 (S)-(2-benzolyphenylamino)-3-{4-[2-(5-methyl-2-phenyl-oxazol-4-yl)ethoxy]phenyl}propionic acid.

Insulin sensitisers also include thiazolidinedione insulin sensitisers.

Other suitable thiazolidinedione insulin sensitisers include (+)-5-[[4-[(3,4-dihydro-6-hydroxy-2,5,7,8-tetramethyl-2H-1-benzopyran-2-yl)methoxy]phenyl]methyl]-2,4-thiazolidinedione (or troglitazone), 5-[4-[(1-methylcyclohexyl)methoxy]benzyl]thiazolidine-2,4-dione (or ciglitazone), 5-[4-[2-(5-ethylpyridin-2-yl)ethoxy]benzyl]thiazolidine-2,4-dione (or pioglitazone) or 5-[(2-benzyl-2,3-dihydrobenzopyran)-5-ylmethyl)thiazolidine-2,4-dione (or englitazone).

Particular thiazolidinedione insulin sensitisers are 5-[4-[2-(5-ethylpyridin-2-yl) ethoxy]benzyl]thiazolidine-2,4-dione (or pioglitazone) and (+)-5-[[4-[(3,4-dihydro-6-hydroxy-2,5,7,8-tetramethyl-2H-1-benzopyran-2-yl)methoxy]phenyl]methyl]-2,4-thiazolidinedione (or troglitazone).

A preferred thiazolidinedione insulin sensitiser is 5-[4-[2-(N-methyl-N-(2-pyridyl) amino)ethoxy]benzyl]thiazolidine-2,4-dione (or rosiglitazone) and salts thereof.

Further antidiabetic agents include other inhibitors of DP IV. Particular DP IV-inhibitors include the specific examples disclosed in WO 99/61431, such as L-threo-isoleucyl pyrrolidide, L-allo-isoleucyl thiazolidide, L-alloisoleucyl pyrrolidide and salts thereof. A particular DP IV-inhibitor is isoleucine thiazolidide and salts thereof.

Further DP IV-inhibitors include valine pyrrolidide (Novo Nordisk), NVP-DPP728A (1-[[[2-[{5-cyanopyridin-2-yl}amino]ethyl]amino]acetyl]-2-cyano-(S)-pyrrolidine) (Novartis) as disclosed by Hughes et al., Biochemistry, 38 (36), 11597-11603, 1999, LAF-237 (1-[(3-hydroxy-adamant-1-ylamino) acetyl]pyrrolidine-2(S)-carbonitrile); disclosed by Hughes et al., Meeting of the American Diabetes Association 2002, Abstract no. 272 or (Novartis), TSL-225 (tryptophyl-1,2,3,4-tetrahydroisoquinoline-3-carboxylic acid), disclosed by Yamada et.al., Bioorg. & Med. Chem. Lett. 8 (1998), 1537-1540, 2-cyanopyrrolidides and 4-cyanopyrrolidides as disclosed by Asworth et al., Bioorg. & Med. Chem. Lett., 6, No. 22, pp 1163-1166 and 2745-2748 (1996), FE-999011 ([(2S)-1-([2′S]-2′-amino-3′,3′dimethylbutanoyl)pyrrolidine-2-carbonitrile]), disclosed by Sudre et al., Diabetes 51 (5), pp 1461-1469 (2002) (Ferring) and the compounds disclosed in WO 01/34594 (Guilford), employing dosages as set out in the above references.

For the avoidance of doubt, the examples disclosed in each of the above mentioned publications are specifically incorporated herein by reference in their entirety, as individually disclosed compounds, especially concerning their structure, their definition, uses and their production.

Preferred embodiments of the present invention comprise the use of compounds of formula (I), or pharmaceutically acceptable salts thereof, according to any one of the embodiments of the present invention:
in combination with acarbose, or
in combination with metfomin; or
in combination with acarbose and metformin; or
in combination with an insulin sensitizer, e.g. a PPARy agonist insulin sensitiser.

The use of a compound of formula (I), or a pharmaceutically acceptable salt thereof, in particular glutaminyl thiazolidine hydrochloride, in combination with metformin e.g. for the treatment of diabetes mellitus, conditions associated with diabetes mellitus and conditions associated with the pre-diabetic state, is especially preferred according to the present invention. The compound of formula (I), or a pharmaceutically acceptable salt thereof, and metformin are preferably co-administered.

The further preferred aspect of the invention is a pharmaceutical composition comprising glutaminyl thiazolidine or glutaminyl pyrrolidine, or a pharmaceutically acceptable salt thereof, in particular glutaminyl thiazolidine hydrochloride, and metformin, and a pharmaceutically acceptable carrier. The pharmaceutical formulation is preferably adapted for oral administration and in particular is in unit does form adapted for administration once, twice or three times, preferably twice or three times, a day.

The use of a compound of formula (I), or a pharmaceutically acceptable salt thereof, in particular glutaminyl thiazolidine hydrochloride, in combination with an insulin sensitiser e.g. a PPARy agonist insulin sensitiser represents a further preferred aspect of the invention. Particular insulin sensitisers include the glitazones e.g. troglitazone, ciglitazone, pioglitazone, englitazone and rosiglitazone, in particular rosiglitazone.

It will be understood that the compounds of formula (I), or pharmaceutically acceptable salts thereof, and the other antidiabetic agents are each administered in a pharmaceutically acceptable form, including pharmaceutically acceptable derivatives such as pharmaceutically acceptable salts, esters and solvates thereof, as appropriate of the relevant pharmaceutically active agent. In certain instances herein the names used for the other antidiabetic agent may relate to a particular pharmaceutical form of the relevant active agent. It will be understood that the use of all pharmaceutically acceptable forms of the active agents per se is encompassed by this invention.

The compounds of formula (I) and pharmaceutically acceptable salts thereof, possess several unexpected characteristics compared to other DP IV-inhibitors already known in the art, which may provide them with certain advantages when administered in combination with other antidiabetic agents according to the invention. These characteristics include, for example:
no activity against non-DP IV and non-DP IV-like enzymes, e.g. DP I, prolyl oligopeptidase, prolidase (see example 12);
high stability in isolated human plasma in vitro (see example 13);
a completely new and controllable mechanism of inactivation/metabolism of the glutamine moiety to the respective pyroglutaminyl compound in vivo, resulting in a shorter half-life than other DP IV inhibitors (see example 8); and
a presumably non-liver dependent half-life in vivo.

Pharmaceutically acceptable salts of the compounds of formula (I) include acid addition salts, i.e. where the amino acid basic side chain is protonated with an inorganic or organic acid. Representative organic or inorganic acids include hydrochloric, hydrobromic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toulenesulfonic, cyclohexanesulfamic, salicylic, saccharinic, trifluoroacetic, sulfinic and 3,5-di-tert-butylbenzoic acid. The use of all pharmaceutically acceptable acid addition salt forms of the compounds of formula (I) is embraced by the scope of this invention.

Preferred acid addition salts of the compounds of formula (I) are the fumarate, benzoate, maleinate, oxalate, 3,5-di-tertiary-butylbenzoate, salicylate, acetate and hydrochloride salts (see example 14). The most preferred acid addition salt of the compounds of formula (I) is the hydrochloride salt. The preferred compound of formula (I) being glutaminyl thiazolidine hydrochloride.

For the avoidance of doubt whenever a compound of formula (I) is referred to in the context of the present invention it is to be understood that reference is being made to both the free base and the corresponding salts, provided such is possible or appropriate under the circumstances.

The present invention further includes within its scope the use of prodrugs of the compounds of formula (I). In general, such prodrugs will be functional derivatives of the compounds which are readily convertible in vivo into the desired therapeutically active compound. Thus, in these cases, the methods of treatment of the present invention, the term “administering” shall encompass the treatment of the various disorders described with prodrug versions of the compounds of formula (I) which converts to the specified compound in vivo after administration to the subject. Procedures for the selection and preparation of suitable prodrug derivatives are described, for example, in “Design of Prodrugs”, ed. H. Bundgaard, Elsevier, 1985. Specific prodrugs are described in patent applications DE 198 28 113, DE 198 28 114, WO 99/67228 and WO 99/67279.

Where the compounds of formula (I) have at least one chiral center, they may accordingly exist as enantiomers. In the case of compounds, e.g. prodrugs, which possess two or more chiral centers, they may additionally exist as diastereomers. It is to be understood that all such isomers and mixtures thereof are encompassed within the scope of the present invention.

Where the processes for the preparation of the compounds of formula (I) give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their components enantiomers by standard techniques, such as the formation of diastereomeric pairs by salt formation with an optically active acid, such as (−)-di-p-toluoyl-d-tartaric acid and/or (+)-di-p-toluoyl-1-tartaric acid followed by fractional crystallization and regeneration of the free base. The compounds may also resolved by formation of diastereomeric esters or amides, followed by chromatographic separation and removal of the chiral auxiliary. Alternatively, the compounds may be resolved using a chiral HPLC column.

Where the compounds of formula (I) are preferably have L-alpha-glutylamine derivatives.

During any of the processes for preparation of the compounds of formula (I), it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using conventional methods known from the art.

Furthermore, some of the crystalline forms of the compounds of formula (I) may exist as polymorphs and as such are included in the present invention. In addition, some of the compounds may form solvates with water (i.e. hydrates) or common organic solvents, and such solvates are also intended to be encompassed within the scope of this invention.

The compounds of formula (I), and pharmaceutically acceptable salts thereof, can also be obtained in the form of their hydrates, or include other solvents used for their crystallization.

As indicated above, the compounds of formula (I), and pharmaceutically acceptable salts thereof, are useful in inhibiting DP IV and DP IV-like enzyme activity. The ability of the compounds of formula (I), and pharmaceutically acceptable salts thereof, to inhibit DP IV and DP IV-like enzyme activity may be demonstrated employing the DP IV activity assay for determination of the Kᵢ-values in vitro and in human plasma, as described in examples 4 and 5. The Kᵢ-values of the compounds of the present invention were determined for glutaminyl thiazolidine as Kᵢ=3.12*10⁻⁷ M±5.11*10⁻¹⁰ M and for glutaminyl pyrrolidine as Kᵢ=1.30*10⁻⁶ M±8.49*10⁻⁸ M against porcine kidney DP IV. The Kᵢ-values of the compounds of the present invention were determined for glutaminyl thiazolidine as Kᵢ=4.03*10⁻⁷ M±2.19*10⁻¹⁰ M after 5 min 5.13*10⁻⁷ M±1.26*10⁻⁸ M after 22 hours pre-incubation, and for glutaminyl pyrrolidine as Kᵢ=1.30*10⁻⁶ M±4.89*10⁻⁸ M after 5 min and 1.36*10⁻⁶ M±3.21*10⁻⁸ M after 22 hours pre-incubation in human plasma.

The ability of the compounds of formula (I), and pharmaceutically acceptable salts thereof, to inhibit DP IV in vivo may be demonstrated by oral or intravasal administration to Wistar rats, as described in example 9. The compounds inhibit DP IV activity in vivo after both, oral and intravasal administration to Wistar rats.

The compounds of formula (I), and pharmaceutically acceptable salts thereof, are able to inhibit DP IV in vivo.

The compounds of formula (I) and pharmaceutically acceptable salt, thereof improve glucose tolerance by lowering elevated blood glucose levels in response to an oral glucose challenge and, therefore, are useful in treating non-insulin-dependent diabetes mellitus. The ability of the compounds of formula (I), and pharmaceutically acceptable salts therof, to improve glucose tolerance in response to an oral glucose challenge, may be measured in diabetic Zucker rats. The method is described in examples 6 and 7. Oral administration of 5 mg/kg b.w., 15 mg/kg and 50 mg/kg b.w. glutaminyl thiazolidine or glutaminyl pyrrolidine resulted in a dose dependent lowering of elevated blood glucose levels and thereby in an improvement of glucose tolerance in diabetic Zucker rats.

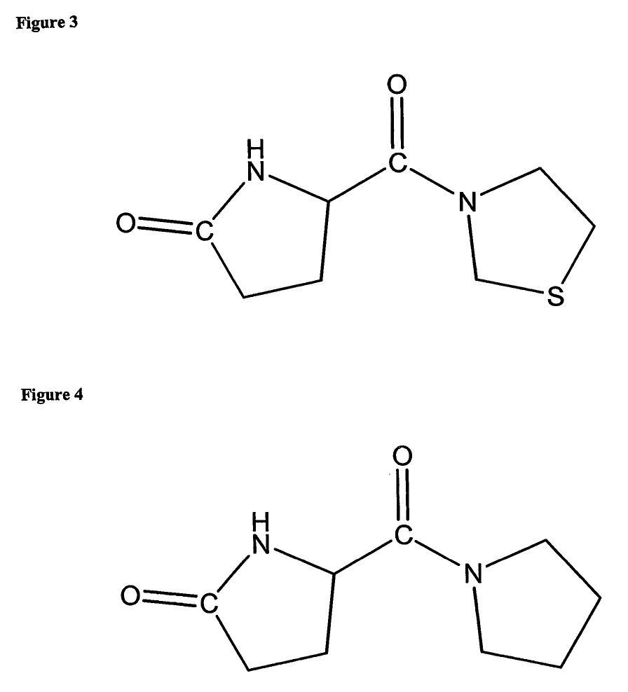

Surprisingly, the compounds of formula (I), and pharmaceutically acceptable salts thereof, are degraded in vivo in a controllable manner following administration to a mammal. The ability of the compounds of formula (I), and pharmaceutically acceptable salts thereof, to be degraded in vivo may be determined employing the Wistar rat model and subsequent LC/MS analysis (see example 8). Glutaminyl thiazolidine and glutaminyl pyrrolidine were found to be degraded following oral administration to Wistar rats, to pyroglutaminyl thiazolidine (FIG. 3) and pyroglutaminyl pyrrolidine (FIG. 4), respectively.

A further embodiment of the present invention comprises the use of compounds of formula (I), or pharmaceutically acceptable salts thereof, according to any one of the embodiments of the present invention mentioned above:

in combination with a gene therapeutic expression system for GLP-1 comprising a viral vector comprising
(a) a polynucleotide sequence encoding GLP-1 (gluacogen like peptide-1); and
(b) a polynucleotide sequence encoding a signal sequence upstream of (a); and
(c) a polyadenylation signal downstream of (a); and
(d) a polynucleotide sequence encoding a proteolytic cleavage site located between the polynucleotide sequence encoding GLP-1 and the polynucleotide sequence encoding the signal sequence; and
(e) wherein the expression of GLP-1 underlies a constitutive promoter or is controlled by a regulatable promotor;
(f) wherein, optionally, the viral vector comprises a polynucleotide sequence encoding GIP (glucose dependent insulinotropic peptide);
(g) wherein, optionally, the viral vector is encompassed by a mammalian cell.
   and/or

in combination with a gene therapeutic expression system for GIP comprising a viral vector comprising
(a) a polynucleotide sequence encoding GIP (glucose dependent insulinotropic peptide); and
(b) a polynucleotide sequence encoding a signal sequence upstream of (a); and
(c) a polyadenylation signal downstream of (a); and
(d) a polynucleotide sequence encoding a proteolytic cleavage site located between the polynucleotide sequence encoding GIP and the polynucleotide sequence encoding the signal sequence; and
(e) wherein the expression of GIP underlies a constitutive promoter or is controlled by a regulatable promotor;
(f) wherein, optionally, the viral vector comprises a polynucleotide sequence encoding GLP-1 (glucagon like peptide-1);
(g) wherein, optionally, the viral vector is encompassed by a mammalian cell.

A further embodiment of the present invention comprises the use of compounds of formula (I), or pharmaceutically acceptable salts thereof, in combination with a gene therapeutic expression system for GLP-1 and/or GIP according to any one of the embodiments of the present invention mentioned above wherein:
the signal sequence upstream of the gene of interest (GLP-1; GIP) is the murine immunoglobulin κ, signal sequence or the glia monster exendin signal sequence; and/or
the polyadenylation signal downstream of the gene of interest (GLP-1; GIP) is derived from simian viraus 40 (SV 40); and/or the proteolytic cleavage site is cleaved by furin preotease; and/or
the gene delivery vector for expression the gene of interest is an adenoviral, retroviral, leniviral, adeno associated viral vector; and/or
the constitutive promoter is a cytomegalovirus (CMV) promotor, or a Rous sarcoma long-terminal repeat (LTR) sequence, and the SV 40 early gene gene promoter; and the inducible promoter is the Tet-On™/Tet-Off™ system available from Clontech; and/or
the mammalian cell is a primate or rodent cell, preferably a human cell, more preferably a human hepatocyte.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human, being sought by a researcher, veterinarian, medical doctor or other clinician, which includes alleviation of the symptoms of the disease or disorder being treated.

When used herein the term “conditions associated with diabetes” includes those conditions associated with the pre-diabetic state, conditions associated with diabetes mellitus itself and complications associated with diabetes mellitus.

When used herein the term “conditions associated with the pre-diabetic state” includes conditions such as insulin resistance, including hereditary insulin resistance, impaired glucose tolerance and hyperinsulinaemia.

“Conditions associated with diabetes mellitus” itself include hyperglycaemia, insulin resistance, including acquired insulin resistance and obesity. Further conditions associated with diabetes mellitus itself include hypertension and cardiovascular disease, especially atherosclerosis and conditions associated with insulin resistance. Conditions associated with insulin resistance include polycystic ovarian syndrome and steroid induced insulin resistance and gestational diabetes.

“Complications associated with diabetes mellitus” includes renal disease, especially renal disease associated with Type 2 diabetes, neuropathy and retinopathy.

Renal diseases associated with Type 2 diabetes include nephropathy, glomerulonephritis, glomerular sclerosis, nephrotic syndrome, hypertensive nephrosclerosis and end stage renal disease.

As used herein, the term “pharmaceutically acceptable” embraces both human and veterinary use: for example the term “pharmaceutically acceptable” embraces a veterinarily acceptable compound or a compound acceptable in human medicine a health care.

To prepare the pharmaceutical compositions of this invention, the compounds of formula (I) or pharmaceutically acceptable salts thereof, optionally in combination with at least on other antidiabetic agent, can be used as the active ingredient(s). The active ingredient(s) is intimately admixed with a pharmaceutical carrier according to conventional pharmaceutical compounding techniques, which carrier may take a wide variety of forms depending of the form of preparation desired for administration, e.g. oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included.

Injectable suspensions may also prepared, in which case appropriate liquid carriers, suspending agents and the like may be employed. The pharmaceutical compositions herein will contain, per dosage unit, e.g. tablet, capsule, powder, injection, teaspoonful and the like, an amount of the active ingredient(s) necessary to deliver an effective dose as described above. The pharmaceutical compositions herein will contain, per dosage unit, e.g., tablet, capsule, powder, injection, suppository, teaspoonful and the like, from about 0.03 mg to 100 mg/kg (preferred 0.1-30 mg/kg) and may be given at a dosage of from about 0.1-300 mg/kg per day (preferred 1-50 mg/kg per day) of each active ingredient or combination thereof. The dosages, however, may be varied depending upon the requirement of the patients, the severity of the condition being treated and the compound being employed. The use of either daily administration or post-periotic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 500 mg of each active ingredient or combinations thereof of the present invention.

The tablets or pills of the compositions of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

This liquid forms in which the compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinylpyrrolidone or gelatin.

The method of treating diabetes mellitus, conditions associated with diabetes mellitus and conditions associated with the pre-diabetic state, as described in the present invention, may also be carried out using a pharmaceutical composition comprising a compound of formula (I), or a pharmaceutically acceptable salt thereof, optionally in combination with at least one other antidiabetic agent or any other of the compounds as defined herein and a pharmaceutically acceptable carrier. The pharmaceutical composition may contain between about 0.01 mg and 100 mg, preferably about 5 to 50 mg, of each compound, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, the compounds of formula (I) and pharmaceutically acceptable salts thereof, may be administered in a single daily dose, or the total daily dosage may be administered in divided doses of two, three or four times daily. Furthermore, the compounds can be administered in intranasal form via topical use of suitable intranasal vehicles, or via transdermal skin patches well known to those of ordinary skill in that art. To be administered in the form of transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or betalactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitable flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

The compounds of formula (I) and the combinations of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines.

The compounds of formula (I) and the combinations of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamidephenol, polyhydroxyethylaspartamid-ephenol, or polyethyl eneoxidepolyllysine substituted with palmitoyl residue. Furthermore, the compounds of the present invention may be coupled to a class of biodegradable polymers useful in achieving controlled release of a drug, for example, polyactic acid, polyepsilon caprolactone, polyhydroxy butyeric acid, polyorthoesters, polyacetals, polydihydropyrans, polycyanoacrylates and cross-linked or amphipathic block copolymers of hydrogels.

The compounds of formula (I) and the combinations of this invention may be administered in any of the foregoing compositions and according to dosage regimens established in the art whenever treatment of the addressed disorders is required.

The daily dosage of the products may be varied over a wide range from 0.01 to 1,000 mg per mammal per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of each active ingredient or combinations thereof for the symptomatic adjustment of the dosage to the patient to be treated. An effective amount of the drug is ordinarily supplied at a dosage level of from about 0.1 mg/kg to about 300 mg/kg of body weight per day. Preferably, the range is from about 1 to about 50 mg/kg of body weight per day. The compounds or combinations may be administered on a regimen of 1 to 4 times per day.

Optimal dosages to be administered may be readily determined by those skilled in the art, and will vary with the particular compound used, the mode of administration, the strength of the preparation, the mode of administration, and the advancement of disease condition. In addition, factors associated with the particular patient being treated, including patient age, weight, diet and time of administration, will result in the need to adjust dosages.

The compounds of formula (I), and pharmaceutically acceptable salts thereof, and the other antidiabetic agent are preferably administered orally.

Suitably, the particularly beneficial effect on glycaemic control provided by the treatment of the invention is an improved therapeutic ratio for the combination of the invention relative to the therapeutic ratio for one compound of the combination when used alone and at a dose providing an equivalent efficacy to the combination of the invention.

In a preferred aspect, the particularly beneficial effect on glycaemic control provided by the treatment of the invention may be indicated to be a synergistic effect relative to the control expected from the effects of the individual active agents.

In a further aspect of the invention, combining doses of the compounds of formula (I), or pharmaceutically acceptable salts thereof, and the other antidiabetic agents may produce a greater beneficial effect than can be achieved for either agent alone at a dose twice that used for that agent in the combination.

Glycaemic control may be characterised using conventional methods, for example by measurement of a typically used index of glycaemic control such as fasting plasma glucose or glycosylated haemoglobin (HbA1c). Such indices are determined using standard methodology, for example those described in: Tuescher A, Richterich, P., Schweiz. med. Wschr. 101 (1971), 345 and 390 and Frank P., ‘Monitoring the Diabetic Patent with Glycosolated Hemoglobin Measurements’, Clinical Products 1988.

The dosage level of each of the active agents when used in accordance with the methods of the invention may be less than would have been required from a purely additive effect upon glycaemic control.

The methods of the invention may also effect an improvement, relative to the individual agents, in the levels of advanced glycosylation end products (AGEs), and serum lipids including total cholesterol, HDL-cholesterol, LDL-cholesterol including improvements in the ratios thereof, in particular an improvement in serum lipids including total cholesterol, HDL-cholesterol, LDL-cholesterol including improvements in the ratios thereof.

In a further aspect, the invention also provides a process for preparing a pharmaceutical composition comprising a compound of formula (I), or a pharmaceutically acceptable salt thereof, another antidiabetic agent and a pharmaceutically acceptable carrier therefor, which process comprises admixing the compound of formula (I), or a pharmaceutically acceptable salt thereof, another antidiabetic agent and a pharmaceutically acceptable carrier.

The compositions are preferably in a unit dosage form in an amount appropriate for the relevant daily dosage.

Suitable dosages, including especially unit dosages, of the compounds of formula (I) or the other antidiabetic agent include the known dosages including unit doses for these compounds as described or referred to in reference text such as the British and US Pharmacopoeias, Remington's Pharmaceutical Sciences (Mack Publishing Co.), Martindale The Extra Pharmacopoeia (London, The Pharmaceutical Press) (for example see the 31st Edition page 341 and pages cited therein) or the above mentioned publications.

Thus, suitable dosages for the compounds of formula (I) include those disclosed therein, for example 0.01 to 30 mg per day or 0.01 to 10 mg per kilogram of body weight. Also, the suitable doses of the other DP IV inhibitors mentioned herein include those mentioned in the relevant publications mentioned above.

For the alpha glucosidase inhibitor, a suitable amount of acarbose is in the range of from 25 to 600 mg, including 50 to 600 mg, for example 100 mg or 200 mg.

For the biguanide, a suitable dosage of metformin is between 100 to 3000 mg, for example 250, 500 mg, 850 mg or 1000 mg.

For the insulin secretagogue, a suitable amount of glibenclamide is in the range of from 2.5 to 20 mg, for example 10 mg or 20 mg; a suitable amount of glipizide is in the range of from 2.5 to 40 mg; a suitable amount of gliclazide is in the range of from 40 to 320 mg; a suitable amount of tolazamide is in the range of from 100 to 1000 mg; a suitable amount of tolbutamide is in the range of from 1000 to 3000 mg; a suitable amount of chlorpropamide is in the range of from 100 to 500 mg; and a suitable amount of gliquidone is in the range of from 15 to 180 mg. Also a suitable amount of glimepiride is 1 to 6 mg and a suitable amount of glipentide is 2.5 to 20 mg.

A suitable amount of repaglinide is in the range of from 0.5 mg to 20 mg, for example 16 mg. Also a suitable amount of nateglinide is 90 to 360 mg, for example 270 mg.

In one particular aspect, the composition comprises 2 to 12 mg of 5-[4-[2-(N-methyl-N-(2-pyridyl) amino)ethoxy]benzyl]thiazolidine-2,4-dione.

Suitable unit dosages of other insulin sensitisers include from 100 to 800 mg of troglitazone such as 200, 400, 600 or 800 mg or from 5 to 50 mg, including 10 to 40 mg, of pioglitazone, such as 20, 30 or 40 mg and also including 15, 30 and 45 mg of pioglitazone.

Suitable dosages of other PPARy agonist insulin sensitisers include those disclosed for the respective agonist in the abovementioned applications, for example 2-(1-carboxy-2-{4-{2-(5-methyl-2-phenyloxazol-4-yl) ethoxy]phenyl}ethylamino)benzoic acid methyl ester and 2(S)-(2-benzoylphenylamino)-3-{4-[2-(5-methyl-2-phenyloxazol- 4-yl)ethoxy]phenyl}propionic acid are suitably dosed in accordance with the dosages disclosed in WO 97/31907.

Also, the dosages of each particular active agent in any given composition can as required vary within a range of doses known to be required in respect of accepted dosage regimens for that compound. Dosages of each active agent can also be adapted as required to take into account advantageous effects of combining the agents as mentioned herein.

The compounds of formula (I) or the compositions of the invention may be taken before a meal, while taking a meal or after a meal.

When taken before a meal the compounds of formula (I) or the compositions of the invention can be taken 1 hour, preferably 30 or even 15 or 5 minutes before eating.

When taken whilst eating, the compounds of formula (I) or the compositions of the invention can be mixed into the meal or taken in a separate dosage form as described above.

When taken after a meal, the compounds of formula (I) or the compositions of the invention can be taken 5, 15 or 30 minutes or even 1 hour after finishing a meal.

No adverse toxicological effects are expected for the compositions or methods of the invention in the above mentioned dosage ranges.

All publications, including, but not limited to, patents and patent application cited in this specification, are herein incorporated by reference as if each individual publication were specifically and individually indicated to be incorporated by reference herein as fully set forth.

The invention is illustrated, but not limited by, the following examples.

### EXAMPLES

### Example 1

### Synthesis of Glutaminyl Pyrrolidine Free Base

N-Benzyloxycarbonylglutamine (2.02 g, 7.21 mmol) was dissolved in 35 mL THF and cooled to −15° C. CAIBE (isobutylchloroformate) (0.937 mL, 7.21 mmol) and 4-methylmorpholine (0.795 mL, 7.21 mmol) were added and the solution stirred for 15 min. The formation of the mixed anhydride was checked by TLC (eluent: CHCl₃/MeOH: 9/1). After warming to −10° C. pyrrolidine (0.596 mL, 7.21 mmol) was added. The mixture was brought to room temperature and stirred overnight. The sediment formed was filtered off and the solvent was evaporated. The resulting oil was taken up in ethylacetate (20 mL) and washed with a saturated solution of sodium hydrogensulfate followed by a saturated solution of sodium bicarbonate, water and brine. The organic layer was separated, dried and evaporated. The resulting product was checked for purity by TLC (eluent: CHCl₃/MeOH: 9/1). Yield: 1.18 g. This product was dissolved in absolute ethanol (40 mL). Into the solution ca. 20 mg Pd on charcoal (10%, FLUKA) was added and the suspension was shaken under a hydrogen atmosphere for 3 h. The progress of the reaction was monitored by TLC (eluent: CHCl₃/MeOH: 9/1). After completion of the reaction the catalyst and solvent were removed to give the title compound (99%). The purity was checked by means of TLC: n-butanol/AcOH/water/ethylacetate: 1/1/1/1, R_{f}=0.4. The identity of the reaction product was checked by NMR analysis.

### Example 2

### Synthesis of Glutaminyl Thiazolidine Hydrochloride

N-t-Butyloxycarbonylglutamine (2.0 g, 8.12 mmol) was dissolved in TBF (5 mL) and cooled to −15° C. CAIBE (isobutylchloroformate) (1.06 mL, 8.12 mmol) and 4-methylmorpholine (0.895 mL, 8.12 mmol) were added and the solution stirred for 15 min, The formation of the mixed anhydride was checked by TLC (eluent: CHCl₃/MeOH: 9/1). After warming to −10° C. another equivalent of 4-methylmorpholine (0.895 mL, 8.12 mmol) and thiazolidinehydrochloride (1.02 g, 8.12 mmol) was added. The mixture was brought to room temperature and stirred overnight. The sediment formed was filtered off and the solvent was evaporated. The resulting oil was taken up in chloroform (20 ml) and washed with a saturated solution of sodium hydrogensulfate followed by a saturated solution of sodium bicarbonate, water and brine. The organic layer was separated, dried and evaporated. The resulting product was checked for purity by TLC (eluent: CHCl₃/MeOH: 9/1). Yield: 1.64 g. A portion of this product (640 mg) was dissolved in 3.1 mL ice cold HCl in dioxane (12.98 M, 20 equivalents) and left on ice. The progress of the reaction was monitored by TLC (eluent: CHCl₃/MeOH: 9/1). After completion of the reaction the solvent was removed and the resulting residue was taken up in methanol and evaporated again. The resulting oil was dried over phosphorous-V-oxide and triturated twice with diethylether to give the title compound (0.265 g). The purity was checked by HPLC. The identity of the reaction product was checked by NMR analysis.

### Example 3

### Synthesis of Glutaminyl Pyrrolidine Hydrochloride

N-t-Butyloxycarbonylglutamine (3.0 g, 12.18 mmol) was dissolved in THF (7 mL) and cooled to −15° C. CAIBE (isobutylchloroformiate) (1.6 mL, 12.18 mmol) and 4-methylmorpholine (1.3 mL, 12.18 mmol) were added and the solution stirred for 15 min. The formation of the mixed anhydride was checked by TLC (eluent: CHCl₃/MeOH: 9/1). After warming to −10° C. 1 equivalent of pyrrolidine (1.0 mL, 12.18 mmol) was added. The mixture was brought to room temperature and stirred overnight. The sediment formed was filtered off and the solvent evaporated. The resulting residue was taken up in chloroform (20 mL) and washed with a saturated solution of sodium hydrogensulfate followed by a saturated solution of sodium bicarbonate, water and brine. The organic layer was separated, dried and evaporated. The resulting product was checked for purity by TLC (eluent: CHCl₃/MeOH: 9/1). The resulting solid (2.7 g) was dissolved in 13.0 mL ice cold HCl in dioxane (12.98 M, 20 equivalents) and left on ice. The progress of the reaction was monitored by TLC (eluent: CHCl₃/MeOH: 9/1). After completion of the reaction the solvent was removed and the resulting residue was taken up in methanol and evaporated again. The resulting residue was dried over phosphorous-V-oxide and triturated twice with diethylether to give the title compound (980 mg). The purity was checked by HPLC. The identity of the reaction product was checked by NMR analysis.

### Example 4

### Kᵢ-Determination

For Kᵢdetermination of glutaminyl pyrrolidine and glutaminyl thiazolidine, dipeptidyl peptidase IV from porcine kidney with a specific activity against glycylprolyl-4-nitroaniline of 37.5 U/mg and an enzyme concentration of 1.41 mg/mL in the stock solution was used.

100 μL glutaminyl pyrrolidine or glutaminyl thiazolidine in a concentration range of 1*10⁻⁵ -1*10⁻⁷ M (glutaminyl pyrrolidine) and 1*10⁻⁶ M-1*10⁻⁸ M (glutaminyl thiazolidine) respectively were admixed with 50 μL glycylprolyl-4-nitroaniline in different concentrations (0.4 mM, 0.2 mM, 0.1 mM, 0,05 mM) and 100 μl HEPES (40 mM, pH7.6; ion strength=0.125). The assay mixture was pre-incubated at 30 ° C. for 30 min. After pre-incubation, 20 μL DPIV (1:600 diluted) were added and measurement of yellow color development due to 4-nitroaniline release was performed at 30° C. and λ=405 nm for 10 min using a plate reader (HTS7000 plus, Applied Biosystems, Weiterstadt, Germany). The Kᵢ-values were calculated using Graphit 4.0.15 (Erithacus Software, Ltd, UK) based on a competitive inhibition of DPIV by glutaminyl pyrrolidine or glutaminyl thiazolidine. They were determined for glutaminyl thiazolidine as Kᵢ=3.12*10⁻⁷ M±5.11*10⁻¹⁰ M and for glutaminyl pyrrolidine as Kᵢ=1.30*10⁻⁶ M±8.49*10⁻⁸ M.

### Example 5

### Kᵢ-Determination in Human Plasma

Human plasma contains N-terminal Xaa-Pro releasing activity. 70 μL glutaminyl pyrrolidine or glutaminyl thiazolidine in an concentration range of 1*10⁻⁵ M -1*10⁻⁷ M (glutaminyl pyrrolidine) and 1*10⁻⁶ M-1*10⁻⁸ M (glutaminyl thiazolidine) respectively were admixed with 50 μL glycylprolyl-4-nitroaniline in different concentrations (0.4 mM, 0.2 mM, 0.1 mM, 0,05 mM) and 100 μl HEPES (40 mM, pH7.6). The assay mixture was pre-incubated at 30 ° C. for 5 min and 22 hours respectively. After pre-incubation, 50 μL human plasma were added and measurement of yellow color development due to 4-nitroaniline release was performed at 30° C. and λ=405 nm for 10 min using a plate reader ([HTS7000 plus, Applied Biosystems, Weiterstadt, Germany). The Kᵢ-values were calculated using Graphit 4.0.15 (Erithacus Software, Ltd, UK) based on a competitive inhibition of DPIV by glutaminyl pyrrolidine or glutaminyl thiazolidine. They were determined for glutaminyl thiazolidine as Kᵢ=4.03*10⁻⁷ M±2.19*10⁻¹⁰ M after 5 min 5.13*10⁻⁷ M±1.26*10⁻⁸ M after 22 hours pre-incubation and for glutaminyl pyrrolidine as Kᵢ=1.30*10⁻⁶ M±4.89*10⁻⁸ M after 5 min and 1.36*10⁻⁶ M±3.21*10⁻⁸ M after 22 hours pre-incubation.

### Example 6

### Dose Escalation Study in Fatty Zucker Rats After Oral Administration of Glutaminyl Pyrrolidine

N=30 male Zucker rats (fa/fa), mean age 11 weeks (5-12 weeks), mean body weight 350 g (150-400 g), were purchased from Charles River (Sulzfeld, Germany). After delivery they were kept for >12 weeks until nearly all fatty Zucker rats had the characteristics of manifest diabetes mellitus. A group of N=8 animals were recruited for testing three escalating doses of glutaminyl pyrrolidine vs. placebo (saline). Animals were single-caged under standardized conditions with controlled temperature (22±2° C.) on a 12/12 hours light/dark cycle (light on at 06:00 AM). Sterile standard pelleted chow (ssniff® Soest, Germany) and tap water acidified with HCl were allowed ad libitum. Fatty Zucker rats of 24-31 weeks (mean: 25 weeks) age, adapted to the housing conditions, were well prepared for the study. Catheters were implanted into the carotid artery of fatty Zucker rats under general anaesthesia (i.p. injection of 0.25 ml/kg b.w. Rompun®[2%], BayerVital, Germany and 0.5 ml/kg b.w. Ketamin 10, Atarost GmbH & Co., Twistringen, Germany). The animals were allowed to recover for one week. The catheters were flushed with heparin-saline (100 IU/ml) three times per week.

Placebo (1 ml saline, 0.154 mol/l) or escalating doses of glutaminyl pyrrolidine (5, 15 and 50 mg/kg b.w.) were administered to groups of N=8 fatty Zucker rats. 375 mg of glutaminyl pyrrolidine were dissolved in 1000 μl DMSO (E. Merck, Darmstadt; Germany [Dimethyl sulfoxide p.a.]). 10 mL saline was added and 1 ml aliquots, each containing 34.09 mg of glutaminyl pyrrolidine, were stored at −20° C. For preparation of the test substance, dose dependent aliquots were diluted in saline. After overnight fasting, placebo or test substance were administered to the fatty Zucker rats via feeding tube orally (15 G, 75 mm; Fine Science Tools, Heidelberg, Germany) at −10 min An oral glucose tolerance test (OGTT) with 2 g/kg b.w. glucose (40% solution, B. Braun Melsungen, Melsungen, Germany) was administered at ±O min via a second feeding tube. Venous blood samples from the tail veins were collected at −30 min, −15 min, ±0 min and at 5, 10, 15, 20, 30, 40, 60, 90 and 120 min into 20 lμl glass capillaries, which were placed in standard tubes filled with 1 mL solution for blood glucose measurement. All blood samples were labelled with Code number, Animal Number, Date of sampling and Time of sampling.

Glucose levels were measured using the glucose oxidase procedure (Super G Glucose analyzer; Dr. Müller Gerältebau, Freital, Germany).

Statistical evaluations and graphics were performed with PRISM® 3.02 (GraphPad Software, Inc.). All parameters were analysed in a descriptive manner including mean and SD.

The placebo treated diabetic Zucker rats showed a strongly elevated blood glucose excursion indicating glucose intolerance of manifest diabetes mellitus. Administration of 5 mg/kg b.w. glutaminyl pyrrolidine resulted in a limited improvement of glucose tolerance in diabetic Zucker rats. Significant lowering of elevated blood glucose levels and improvement of glucose tolerance was achieved after administration of 15 mg/kg and 50 mg/kg b.w. glutaminyl pyrrolidine (see FIG. 3).

### Example 7

### Dose Escalation Study in Fatty Zucker Rats After Oral Administration of Glutaminyl Thiazolidine

N=30 male Zucker rats (fa/fa), mean age 11 weeks (5-12 weeks), mean body weight 350 g (150-400 g), were purchased from Charles River (Sulzfeld, Germany). After delivery they were kept for >12 weeks until nearly all fatty Zucker rats had the characteristics of manifest diabetes mellitus. A group of N=8 animals were recruited for testing three escalating doses of glutaminyl thiazolidine vs. placebo (saline). Animals were single-caged under standardized conditions with controlled temperature (22±2° C.) on a 12/12 hours light/dark cycle (light on at 06:00 AM). Sterile standard pelleted chow (ssniff® Soest, Germany) and tap water acidified with HCl were allowed ad libitum. Fatty Zucker rats of 24-31 weeks (mean: 25 weeks) age, adapted to the housing conditions, were well prepared for the study. Catheters were implanted into the carotid artery of fatty Zucker rats under general anaesthesia (i.p. injection of 0.25 ml/kg b.w. Rompun® [2%], BayerVital, Germany and 0.5 ml/kg b.w. Ketamin 10, Atarost GmbH & Co., Twistringen, Germany). The animals were allowed to recover for one week. The catheters were flushed with heparin-saline (100 IU/ml) three times per week.

Placebo (1 mL saline, 0.154 mol/L) or escalating doses of glutaminyl thiazolidine (5, 15 and 50 mg/kg b.w.) were administered to groups of N=8 fatty Zucker rats. The respective amounts of glutaminyl thiazolidine were dissolved in 1000 μl saline. After overnight fasting, placebo or test substance was administered to the fatty Zucker rats via feeding tube orally (15 G, 75 mm; Fine Science Tools, Heidelberg, Germany) at −10 min An oral glucose tolerance test (OGTT) with 2 g/kg b.w. glucose (40% solution, B. Braun Melsungen, Melsungen, Germany) was administered at ±0 min via a second feeding tube. Venous blood samples from the tail veins were collected at −30 min, −15 min, ±0 min and at 5, 10, 15, 20, 30, 40, 60, 90 and 120 min into 20 μL glass capillaries, which were placed in standard tubes filled with 1 ml solution for blood glucose measurement. All blood samples were labelled with Code number, Animal Number, Date of sampling and Time of sampling.

Glucose levels were measured using the glucose oxidase procedure (Super G Glucose analyzer; Dr. Müiller Gerätebau, Freital, Germany).

Statistical evaluations and graphics were performed with PRISM® 3.02 (GraphPad Software, Inc.). All parameters were analysed in a descriptive manner including mean and SD.

The placebo treated diabetic Zucker rats showed a strongly elevated blood glucose excursion indicating glucose intolerance of manifest diabetes mellitus. Administration of 5 mg/kg b.w., 15 mg/kg and 50 mg/kg b.w glutaminyl thiazolidine resulted in a dose dependent lowering of elevated blood glucose levels and improvement of glucose tolerance in diabetic Zucker rats (see FIG. 4).

### Example 8

### In Vivo Inactivation of Glutaminyl Thiazolidine After Oral Administration to Wistar Rats

Glutaminyl thiazolidine was administered to Wistar rats orally. After application of placebo or glutaminyl thiazolidine, arterial blood samples were taken at 2.5, 5, 7.5, 10, 15, 20, 40, 60 and 120 min from the carotid catheter of the conscious unrestrained rats to determine the formation of degradation products of glutaminyl thiazolidine. For analysis, simple solid phase extraction procedure on C18 cartridges was used to isolate the compounds of interest from the plasma. The extracts were analysed using reversed-phase liquid chromatography on Lichrospher 60 RP Select B column hyphenated with tandem mass spectrometry operating in the APCI positive mode. An internal standard method was used for quantification.

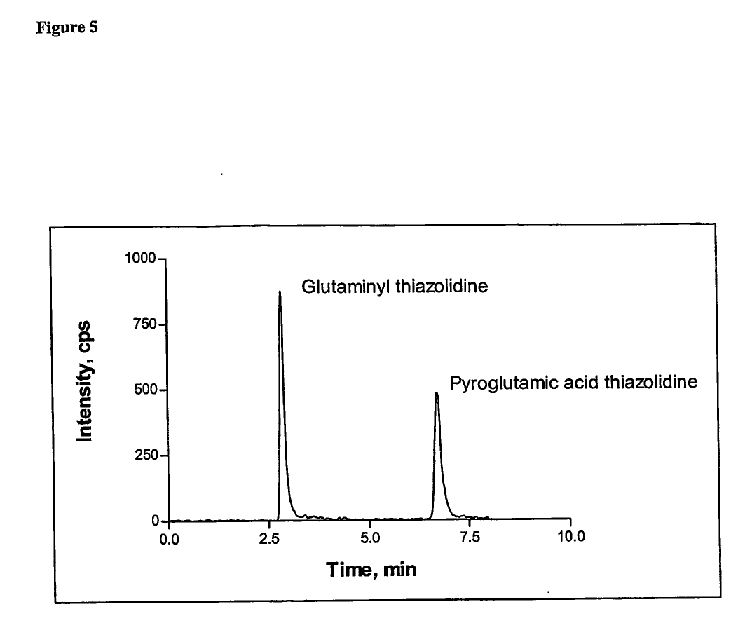

After oral administration of glutaminyl thiazolidine to Wistar rats, a degradation of the compound was found. Using LC/MS, the degradation product could be defined as pyroglutaminyl thiazolidine. See FIGS. 3 and 5.

### Example 9

### Determination of DPIV Inhibiting Activity of Glutaminyl Pyrrolidine and Glutaminyl Thiazolidine After Intravasal and Oral Administration to Wistar Rats

Male Wistar rats (Shoe: Wist(Sho)) with a body weight ranging between 250 and 350 g were purchased from Tierzucht Schönwalde (Schönwalde, Germany). Animals were single-caged under conventional conditions with controlled temperature (22±2° C.) on a 12/12 hours light/dark cycle (light on at 06:00 AM). Standard pelleted chow (ssniff® Soest, Germany) and tap water acidified with HCl were allowed ad libitum. After ≧one week of adaptation at the housing conditions, catheters were implanted into the carotid artery of Wistar rats under general anaesthesia (i.p. injection of 0.25 ml/kg b.w. Rompun®[2%], BayerVital, Germany and 0.5 ml/kg b.w. Ketamin 10, Atarost GmbH & Co., Twistringen, Germany). The animals were allowed to recover for one week. The catheters were flushed with heparin-saline (100 IU/ml) three times per week. In case of catheter dysfunction, a second catheter was inserted into the contra-lateral carotid artery of the respective rat. After one week of recovery from surgery, this animal was reintegrated into the study. In case of dysfunction of the second catheter, the animal was withdrawn from the study. A new animal was recruited and the experiments were continued in the planned sequence, beginning at least 7 days after catheter implantation.

To rats with intact catheter finction were administered placebo (1 mL saline, 0.154 mol/l) or 100 mg/kg b.w. glutaminyl pyrrolidine or 100 mg/kg b.w. glutaminyl thiazolidine via the oral and the intra-vasal (intra-arterial) route. After overnight fasting, 100 μL samples of heparinised arterial blood were collected at −30, −5, and 0 min. The test substance was dissolved freshly in 1.0 mL saline (0.154 mol/l) and was administered at 0 min either orally via a feeding tube (75 mm; Fine Science Tools, Heidelberg, Germany) or via the intra-vasal route. In the case of oral administration, an additional volume of 1 mL saline was injected into the arterial catheter. In the case of intra-arterial administration, the catheter was immediately flushed with 30 μL saline and an additional 1 mL of saline was given orally via the feeding tube. After application of placebo or the test substances, arterial blood samples were taken at 2.5, 5, 7.5, 10, 15, 20, 40, 60 and 120 min from the carotid catheter of the conscious unrestrained rats. All blood samples were collected into ice cooled Eppendorf tubes (Eppendorf-Netheler-Hinz, Hamburg, Germany) filled with 10 μL 1M sodium citrate buffer (pH 3.0) for plasma DPIV activity measurement. Eppendorf tubes were centrifuged immediately (12000 rpm for 2 min, Hettich Zentrifuge EBA 12, Tuttlingen; Germany): The plasma fractions were stored on ice until analysis or were frozen at −20° C. until analysis. All plasma samples were labelled with Code number, Animal Number, Date of sampling and Time of sampling.

The assay mixture for determination of plasma DPIV activity consisted of 80 μL reagent and 20 μL plasma sample. Kinetic measurement of the formation of the yellow product 4-nitroaniline from the substrate glycylprolyl-4-nitroaniline was performed at 390 nm for 1 min at 30° C. after 2 min pre-incubation at the same temperature. The DPIV activity was expressed in mU/mL.

Statistical evaluations and graphics were performed with PRISM® 3.02 (GraphPad Software, Inc.). All parameters were analysed in a descriptive manner including mean and SD.

The compounds glutaminyl pyrrolidine and glutaminyl thiazolidine in a dose of 100 mg/kg b.w. vs. placebo inhibited plasma DPIV activity after oral and intra-vasal administration.

### Example 10

### Effect of Glutaminyl Thiazoildine Hydrochloride and Metformin Either Alone or in Combination on Glycaemic Control in Diet-Induced Obese Rats With Impaired Glucose Tolerance

Selectively bred male rats, 5-6 weeks of age, displaying enhanced likelihood of developing diet-induced obesity (DIO) are selected from the breeding colony. A total of 40 DIO animals are included in the study. The DIO rats are chosen because they are likely to reflect the segment of the human population, who develop obesity and later type diabetes upon exposure to high-calorie fat rich diet.

Upon entry to the experiment, rats are housed individually (1 rat/cage) in a 12/12 light-dark cyldle (light from 0600-1800 h) with controlled temperature conditions (22-24° C.). At this time rats are offered High fat (HF) diet (4.41 kcal/g−Energy %: Carbohydrate 51.4 kcal %, Fat 31.8 kcal %, Protein 16.8 kcal %; diet #12266B; Research Diets, New Jersey, USA; the HF diet ensure sufficient intake of vitamins and trace elements) and water ad libitum. After one week of acclimatisation, 24 h food and water intake and body-weight is measured gravimetrically twice weekly (in the morning between 8-10 am). After 3 weeks of HF feeding, average daily food consumption is calculated for all rats. The average food intake comprises a platform from which a scheduled feeding regime is implemented. Animals are offered 75% of the daily average food consumption from 8:00-12:00 AM, and 25% of the daily average food consumption from 4:00 PM-8:00 PM:

After 3 weeks of schedule feeding, animals are stratified according to weight. At day 0, animals are randomised (n=10 in each group) to participate in one of following drug treatment groups:

| | |
|---|---|
| Group A: | vehicle (distilled water) |
| Group B: | glutaminyl thiazolidine hydrochloride (60 mg/kg BID) |
| Group C: | Metformin (125 mg/kg BID) |
| Group D: | glutaminyl thiazolidine hydrochloride (60 mg/kg BID) + |
| | Metformin (125 mg/kg BID) |

All drugs are given orally by gavage, volume 200 μl, twice daily (8:00 AM and 4:00 PM). This mode of administration ensures that all animals receive the same amount of drug irrespective of the diet eaten thereby ensuring more accurate comparison between the chow and high fat diet fed groups. Animals receive two daily doses of either compound for a total of 42 days (day 1-42). On days 6 and 40 animals are subjected to an oral glucose tolerance test (OGTT). Two days later, on day 42, treatment is discontinued and animals are followed drug free for yet another day (still following the schedule feeding regime). On day 43, animals are sacrificed in a semi-starved state as they have had access to only 25% of their daily energy requirement from 12: AM the previous day. In the morning period (from 8-12 AM), animals are anaesthetised by CO₂ inhalation and blood samples are collected. Optionally, tissue samples can be taken and rapidly frozen in liquid nitrogen for later analysis of tissue specific gene expression and lipid content. Blood and tissue sampling will be carried out in a room adjacent to the permanent stable in order to ensure lowest possible level of stress. Fat samples are weighed and frozen such that accurate analysis of fat depots can be carried out. Fat depot analysis could be carried out by removing mesenterial, retroperitoneal, epididymal and subcutaneous inguinal fat.

Analytical Methods:

Oral Glucose Tolerance Test (OGTT):

This test is carried out at 8:00 AM on days 6 and 42. Animals are mildly fasted as they have had access to only 25% of their daily energy requirements in the preceding 20 hrs (Since 12:00 AM the previous day). Blood samples are taken from an indwelling arterial catheter and P-glucose is measured on automated analyser (Roche Diagnostics) at time points −60, −30, 0, 15, 30, 60, 120, and 180 min after oral administration of 1 g/kg glucose (using 1 g/ml dH₂O). The oral glucose load is given as gavage via a duodenally placed tube connected to a syringe ensuring accurate dosing. P-insulin is measured at time points: 0, 15, 30, 60, 120 using an ultra-sensitive ELISA (Shibayagi,Japan).

Blood Sampling and Plasma Measurements:

All rats are equipped with intra-arterial catheters at day −7. The intra-arterial catheters are positioned in the abdominal aorta via the femoral artery and kept patent by injection of heparinised saline at the end of all sampling procedures. All blood samples are taken in EDTA Vacutainer tubes and plasma glucose is measured together with total Cholesterol and triacylglycerol. Optionally, as a reflection of lipolysis, we could measure plasma levels of glycerol. On the day of sacrifice, heart puncture blood is collected in three tubes: Vacutainer-EDTA; Vacutainer-EDTA+1% NaF; Vacutainer-EDTA+Aprotinin (750 KIU).

Various blood sample “packages” are taken:
A) Glycaemic profile: fasting P-glucose, P-insulin and HbA1c
B) 24 hour glycaemic profile: B-glucose every 3^{rd}hour (8:00, 11:00, 14:00, 17:00, 20:00, 23:00, 02:00, 05:00). Alternatively, P-glucose and P-insulin with same time profile
C) Meal associated glucose: B-glucose before and after morning meal (8:00 AM and 12:00 AM)
D) Fasting glucose & lipids: Fasting-P-glucose, P-triacylglycerol, P-total cholesterol
E) OGTT: for details see above

Plasma-Glucose, HbA1c, Plasma-total Cholesterol, Plasma-triacylglycerol is measured using standard enzyme assay kits on a fully automated analyser (Roche Diagnostics). Plasma non-esterified free fatty acids (NEFA) are determined by a spectrophotometer using acyl-CoA oxidase based colorimetric kit (NEFA-C, WAKO pure chemicals, Osaka, Japan). Samples taken in Vacuatiner-EDTA+1% NaF are used for FFA analyses.

Plasma insulin is measured with an ultra-sensitive ELISA based assay (Shibayagi, Japan). Bioactive GLP-1(7-37) and total GLP-1 immunoreactivities are measured with a Linco multiple ELISA kit (Linco Research Immunoassay, St. Charles, Mo.).

Data, Reporting, and Statistical Evaluation:

All data is fed into Excel 97 or 2000 spread sheets and subsequently subjected to relevant statistical analyses (Statview or Graph Pad software). Results are presented as mean±SEM (standard error of the mean) unless otherwise stated. Statistical evaluation of the data is carried out using one-way analysis of variance (ANOVA) with appropriate post-hoc analysis between control and treatment groups in cases where statistical significance is established (p<0.05).

Results:

Using a protocol of this type both glutaminyl thiazolidine and glutaminyl thiazolidine in combination with Metformin resulted in improved oral glucose tolerance.

### Example 11

### Inhibition of DP IV-Like Enzymes—Dipeptidyl Peptidase II

DP II (3.4.14.2) releases N-terminal dipeptides from oligopeptides if the N-terminus is not protonated (McDonald, J. K., Ellis, S. & Reilly, T. J., 1966, J Biol. Chem., 241, 1494-1501). Pro and Ala in P₁-position are preferred residues. The enzyme activity is described as DPIV-like activity, but DP II has an acidic pH-optimum. The enzyme used was purified from porcine kidney. 100 μL glutaminyl pyrrolidine or glutaminyl thiazolidine in an concentration range of 1*10⁻⁴M-5*10⁻⁸M were admixed with 100 μL buffer solution (40 mM HEPES, pH 7.6, 0.015% Brij, 1 mM DTT), 50 μL lysylalanylaminomethylcoumarine solution (5 mM) and 20 μl porcine DP II (250 fold diluted in buffer solution). Fluorescence measurement was performed at 30° C. and λₑₓᵢₐₜₐₜᵢₒₙ=380 nm, λₑₘᵢₛₛᵢₒₙ=465 nm for 25 min using a plate reader (HTS7000 plus, Applied Biosystems, Weiterstadt, Germany). The Kᵢ-values were calculated using Graphit 4.0.15 (Erithacus Software, Ltd., UK) and were determined as Kᵢ₌8.52*10⁻⁵ M±6.33*10⁻⁶ M for glutaminyl pyrrolidine and Kᵢ₌1.07*10⁻⁵ M±3.81*10⁻⁷ M for glutaminyl thiazolidine.

### Example 12

### Cross Reacting Enzymes

Glutaminyl pyrrolidine or glutaminyl thiazolidine were tested for their cross reacting potency against dipeptidyl peptidase I, prolyl oligopeptidase and prolidase.

Dipeptidyl Peptidase I (DP L Cathepsin C)

DP I or cathepsin C is a lysosomal cysteine protease which cleaves dipeptides from the N-terminus of their substrates (Gutman, H. R. & Fruton, J. S., 1948, J Biol. Chem., 174, 851-858). It is classified as a cysteine protease. The enzyme used was purchased from Qiagen (Qiagen GmbH, Hilden, Germany). In order to get a fully active enzyme, the enzyme was diluted 1000 fold in MES buffer pH5.6 (40 mM MES, 4 mM DTT, 4 mM KCl, 2 mM EDTA, 0.015% Brij) and pre-incubated for 30 min at 30° C. 50 μL glutaminyl pyrrolidine or glutaminyl thiazolidine in a concentration range of 1*10⁻⁵ M-1*10⁻⁷ M were admixed with 110 μL buffer-enzyme-mixture. The assay mixture was pre-incubated at 30° C. for 15 min After pre-incubation, 100 μL histidylseryl-para-nitroanilide (2*10⁻⁵ M) were added and measurement of yellow color development due to para-nitroaniline release was performed at 30° C. and λ_{excitation}=380 nm, λₑₘᵢₛₛᵢₒₙ=465 nm for 10 min, using a plate reader (HTS7000 plus, Applied Biosystems, Weiterstadt, Germany). The IC₅₀-values were calculated using Graphit 4.0.15 (Erithacus Software, Ltd., UK). No inhibition of the DP I enzyme activity by glutaminyl pyrrolidine or glutaminyl thiazolidine was found.

Prolyl Oligopeptidase (POP)

Prolyl oligopeptidase (EC 3.4.21.26) is a serine type endoprotease which cleaves off peptides at the N-terminal part of the Xaa-Pro bond (Walter, R., Shlank, H., Glass, J. D., Schwartz, I. L. & Kerenyi, T. D., 1971, Science, 173, 827-829). Substrates are peptides with a molecular weight up to 3000 Da. The enzyme used was a recombinant human prolyl oligopeptidase. Recombinant expression was performed in E. coli under standard conditions as described elsewhere in the state of the art. 100 μL glutaminyl pyrrolidine or glutaminyl thiazolidine in an concentration range of 1*10⁻⁴ M-5*10⁻⁸ M were admixed with 100 μL buffer solution (40 mM HEPES, pH 7.6, 0.015% Brij, 1 mM DTT) and 20 μL POP solution. The assay mixture was pre-incubated at 30° C. for 15 min After pre-incubation, 50 μL glycylprolylprolyl-4-nitroaniline solution (0.29 mM) were added and measurement of yellow color development due to 4-nitroaniline release was performed at 30° C. and λ=405 nm for 10 min using a plate reader (sunrise, Tecan, Crailsheim, Germany). The IC ₅₀-values were calculated using Graphit 4.0.15 (Erithacus Software, Ltd., UK). No inhibition of POP activity by glutaminyl pyrrolidine or glutaminyl thiazolidine was found.

Prolidase (X-Pro Dipeptidase)

Prolidase (EC 3.4.13.9) was first described by Bergmann & Fruton (Bergmann, M. & Fruton, J. S., 1937, J Biol. Chem. 189-202). Prolidase releases the N-terminal amino acid from Xaa-Pro dipeptides and has a pH optimum between 6 and 9. Prolidase from porcine kidney (ICN Biomedicals, Eschwege, Germany). was solved (1 mg/mL) in assay buffer (20 mM NH₄(CH₃COO)₂, 3 mM MnCl₂, pH 7.6). In order to get a fully active enzyme the solution was incubated for 60 min at room temperature. 450 μL glutaminyl pyrrolidine or glutaminyl thiazolidine in an concentration range of 5*10⁻³ M-5*10⁻⁷ M were admixed with 500 μL buffer solution (20 mM NH₄(CH₃COO)₂, pH 7.6) and 250 μL Ile-Pro-OH (0.5 mM in the assay mixture). The assay mixture was pre-incubated at 30° C. for 5 min After pre-incubation, 75 μL Prolidase (1:10 diluted in assay buffer) were added and measurement was performed at 30° C. and λ=220 nm for 20 min using a UV/Vis photometer, UVW (Thermo Spectronic, Cambridge, UK). The IC₅₀-values were calculated using Graphit 4.0.15 (Erithacus Software, Ltd., UK). They were determined as IC 50>3 mM for glutaminyl thiazolidine and as IC₅₀=3.4*10⁻⁴ M±5.63*10⁻⁵ for glutaminyl pyrrolidine.

### Example 13

### Plasma Stability

In order to investigate the stability of glutaminyl pyrrolidine or glutaminyl thiazolidine in human plasma, the activity of DPIV in plasma was determined at a defined time. The average DPIV activity in human plasma was determined as 43.69 U/mL. In the working solution, the plasma was diluted in 0.9% NaCl to fix the DPIV activity level at 25 U/mL. Plasma and glutaminyl pyrrolidine or glutaminyl thiazolidine in different concentrations (5*10⁻⁵, 2.5*10⁻⁵, 1.25*10⁻⁵ M in plasma) were incubated at 37° C. At defined time points samples were taken using a pipette roboter (Gilson 215, Liquid handler, Gilson) and transferred in a microtiter plate containing 5*10⁻⁵ M glycylprolylaminomethylcoumarine in 0.9% NaCl+015% Brij per well. After 6 min the reaction was stopped by addition of isoleucylthiazolidine (5*10⁻⁵ M in 0.9% NaCl solution). Fluorescence measurement was performed against 0.9% NaCl in plasma (reference standard) using a plate reader (HTS7000 plus, Applied Biosystems, Weiterstadt, Germany). The half-life of the inhibitory potency of glutaminyl pyrrolidine or glutaminyl thiazolidine was calculated by plotting the enzyme activity versus reaction time. For both compounds, no half-time could be determined. The substance is considered to be stable in human plasma over 22 hours.

### Example 14

### Synthesis of Other Salt Forms of Glutaminyl Thiazolidine

Glutaminyl thiazolidine hydrochloride (1 g, 3.43 mmol) was applied on a strong basic ion exchange column (DOWEX® 550 A, 10 mL dry material, preconditioned as described). The fractions were collected and titrated with 1N HCl against bromthymolblue in order to estimate the content of free base. After that the corresponding amount of the required acid was added and the solution was lyophilized. The resulting material was re-crystallized from methanol/ether.

The 3,5-di-tertbutylbenzoate and sulfinate salts of glutaminyl thiazolidine are novel and as such form a fiuther aspect of the present invention.

Characterization of different acid addition salts of glutaminyl thiazdlidine:

| Salt | PGT | MP (° C.) | Appearance |
|---|---|---|---|
| Hydrochloride | <0.5 | 167-169 | crystalline |
| Fumarate | 0.2 | 128-131 | crystalline |
| Benzoate | 0.6 | 116-118 | crystalline |
| Maleinate | 4.6 | 128-132 | crystalline |
| Oxalate | 0 | Broad, | amorphous |
| | | 90-110 | |
| 3,5-Di-tert-butylbenzoate | 1.06 | Sharp, | crystalline |
| | | 125 | |
| Sulfinate | Unknown by- | 143-145 | crystalline |
| | product | | |
| Salicylate | 0.6 | 120-127.6 | crystalline |
| Acetate | 0.1 | 88-89 | amorphous |

PGT pyroglutaminyl thiazolidine, area % determined by HPLC analysis
MP melting point

### Example 15

### Effects of Glutaminyl Thiazolidine and Metformin Either Alone or in Combination on Glycemic Control in Diabetic Zucker (fa/fa) Rats

Ten or eleven weeks old male Zucker (fa/fa) rats were purchased from Charles River (Sulzfeld, Germany). Animals were kept under standardized semi-barrier conditions with controlled temperature (22±2° C.) on a 12/12 hours light/dark cycle (light on at 06:00 a.m.). Standard pelleted chow (ssniff®, Soest, Germany) and tap water acidified with HCl were allowed ad libitum. At the age of 12 weeks the animals (N=42) were divided in random order into six experimental groups to be medicated. Definition of the Experimental Groups for the Medications (GT=glutaminyl thiazolidine):

| | |
|---|---|
| Group CO (N = 7): | placebo (distilled water), b.i.d., oral |
| Group GT (N = 7): | 60 mg/kg b.w. GT, b.i.d., oral |
| Group Met-low (N = 7): | 125 mg/kg b.w. Metformin, b.i.d., oral |
| Group Met-high (N = 7): | 300 mg/kg b.w. Metformin, b.i.d., oral |
| Group GT + Met-low (N = 7): | 60 mg/kg b.w. GT + 125 mg Metformin, |
| | b.i.d., oral |
| Group GT + Met-high (N = 7) | 60 mg/kg b.w. GT + 300 mg Metformin, |
| | b.i.d., oral |

The single or combined doses per kg b.w. were solved in 5 mL dist. water for oral administration.

Experimental procedures:

First OGTT:

The study was started with the 12 to 13 weeks old Zucker (fa/fa) rats. At the beginning of the study an OGTT was performed (2 g glucose/kg body weight (b.w.); administration volume: 5 mL/kg of a 40% solution; B. Braun Melsungen, Melsungen, Germany) after a 16 h fast. The glucose was administered via a feeding tube (15 g, 75 mm; Fine Science Tools, Heidelberg, Germany). Dosing was performed at t=5 min before OGTT by gavage.

Group characterization and medication for the first and second OGTT

| | Animals | | |
|---|---|---|---|
| Group | (N) | Drug/Dose | Comments |
| CO | 7 | Placebo; 5 ml/kg b.w. | 0.5 mL/100 g b.w. |
| GT | 7 | P93/01; 60 mg/kg b.w. | 60 mg P93/01 solved in 5 mL dist. |
| | | | water, 0.5 mL/100 g b.w |
| Met-low | 7 | Metformin; 125 mg/kg b.w. | 125 mg Metformin solved in 5 mL |
| | | | dist. water, 0.5 mL/100 g b.w |
| Met-high | 7 | Metformin; 300 mg/kg b.w. | 300 mg Metformin solved in 5 mL |
| | | | dist. water, 0.5 mL/100 g b.w |
| GT + Met-low | 7 | GT; 60 mg/kg b.w. + Metformin; | 60 mg GT + 125 mg Metformin |
| | | 125 mg/kg b.w. | solved in 5 mL dist. water, |
| | | | 0.5 mL/100 g b.w |
| GT + Met-high | 7 | GT; 60 mg/kg b.w. + Metformin; | 60 mg GT + 300 mg Metformin |
| | | 300 mg/kg b.w. | solved in 5 mL dist. water, |
| | | | 0.5 mL/100 g b.w |

Blood samples were taken from tail veins to measure blood glucose and serum insulin at −15, ±0 min, 15, 30, 60, 90, 120 and 180 min (the latter time without insulin samples) with respect to time of glucose administration.

After the first OGTT the animals in the groups were dosed twice daily with the respective drugs at 08:00 AM and at 04:00 PM, respectively:

During the two weeks of medication morning blood glucose was measured before the 08:00 AM medication at Monday, Wednesday and Friday.

Food intake was determined every day during the time of medication.

All the animals were weighted three times per week at 7:30 AM.

A second OGTT was performed after two weeks of medication (Day 15). The food was withdrawn at 04:00 PM the day before (16 h fast). The OGTT was performed with pre-medication at −5 min and oral glucose loading at ±0 min. Blood samples were taken from tail veins to measure blood glucose, and serum insulinₑ at −15, ±0 min, 15, 30, 60, 90, 120 and 180 min (the latter time without insulin samples).

Glycated hemoglobin was measured before (Day −7) and on Day 18.

Measurements:

Glucose-For determination of glucose 20 μL blood were collected at −15, ±0 min (before OGTT) and 15, 30, 60, 90, 120 and 180 min post OGTT.

Insulin-Insulin concentrations were assayed by the antibody RIA method (Linco Research, Inc. St. Charles, Mo., USA).

Glycated haemoglobin-Percentage of glycated hemoglobin A (HbA1c) was estimated with the “DCA 2000R Hämoglobin A1c-Reagenz kit”(Bayer Vital GmbH, Fernwald, Germany).

The body weight was measured using a platform balance (Scaltec, Heiligenstadt, Germany).

Mixed venous blood samples from the tails were collected into 20 μL glass capillaries, which were placed in standard tubes filled with 1 ml solution for hemolysis (blood glucose measurement) and in sample tubes for serum insulin (50 μL blood).

Raw data of glucose analysis were provided by IDK to probiodrug in Excel format as soon as possible. Data for each drug and each parameter (glucose, insulin) were summarized by descriptive statistics (mean, SEM). AUC and baseline corrected AUC (baseline was set to the value at t=0 min) were calculated. Changes from baseline were calculated and summarized by descriptive statistic.

Results:

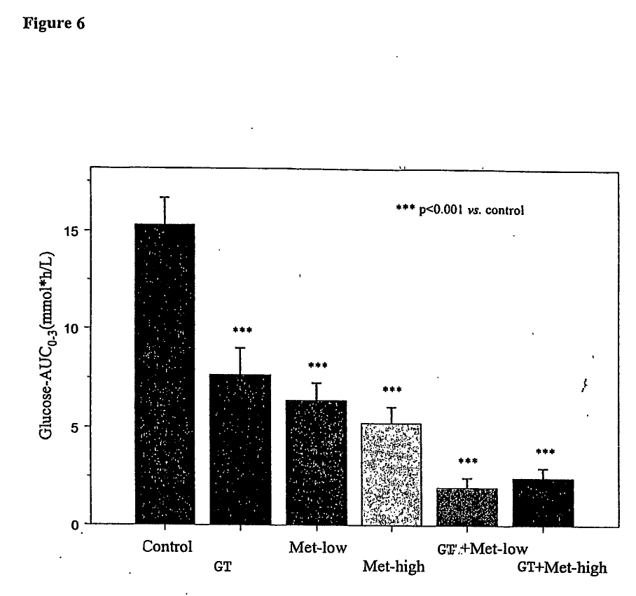

Subchronic (18 days) b.i.d. administration of glutaminyl thiazolidine alone or in combination with Metformin to diabetic fatty Zucker rats (fa/fa) resulted in an improved glucose tolerance. Drug administration had no affected food and water intake in all experimental groups. GT and Met-low groups showed significantly improved glucose tolerance curves in the OGTT and the reactive and absolute G-AUC were significantly reduced (p<0.05 vs. Control). Met-high, GT+Met-low and GT+Met-high groups revealed a further improvement of glucose tolerance curve and the reactive and absolute G-AUC were once more lowered (p<0.05 vs. Control). FIG. 6 shows baseline corrected glucose AUC during first OGTT in fasted Zucker rats loaded with placebo, GT, Met-low, Met-high, GT+Met-low and GT+Met-high (at −5 min) and OGTT (at 0 min) after 14 days of medication (baseline was set as y-value at t=O min).

### Example 16

### Effects of Combination Therapy of Glutaminyl Thiazolidine with Other Oral Antidiabetics

Male, eight weeks old male Zucker (fa/fa) rats were kept under standardized semi-barrier conditions with controlled temperature (22±2° C.) on a 12/12 hours light/dark cycle (light on at 06:00 a.m.). Standard pelleted chow (ssniff®, Soest, Germany) and tap water acidified with HCl were allowed ad libitum. At the age of 12 weeks the animals (N=42) were divided in random order into six experimental groups to be medicated. The experimental groups for the two weeks of medication are as follows (GT=glutaminyl thiazolidine):

| | |
|---|---|
| Group CO (N = 5): | placebo (distilled water), b.i.d., oral at 08.00 AM |
| | and 04.00 PM |
| Group GT (N = 5) | 60 mg/kg b.w. GT b.i.d., oral at 08.00 AM and |
| | 04.00 PM. |
| Group Rosiglitazone + GT (N = 5): | 3 mg/kg b.w. Rosiglitazone, once per day p.o. at |
| | 08.00 AM + 60 mg/kg b.w. GT, b.i.d., oral at 08.00 |
| | AM and 04.00 PM. |
| Group Acarbose + GT (N = 5): | 40 mg acarbose/100 g chow with free (food) access + 60 mg/kg |
| | b.w. GT, b.i.d., oral at 08.00 AM and |
| | 04.00 PM. |
| Group Glibenclamide + GT (N = 5): | 5 mg/kg b.w. glibenclamide, b.i.d., oral + 60 mg/kg |
| | b.w. GT, b.i.d., oral at 08.00 AM and 04.00 PM. |
| Group Insulin + GT (N = 5): | 2 IU long acting insulin b.i.d., SC + 60 mg/kg b.w. |
| | GT, b.i.d., oral at 08.00 AM and 04.00 PM. |

The single or combined oral doses per kg b.w. were solved in 5 mL 1% methylcellulose in saline.

The study was started with the 12 weeks old Zucker (fa/fa) rats. At the beginning of the study a first OGTT was performed (dose: 2 g glucose/kg body weight (b.w.); administration volume: 5 mL/kg of a 40% solution; B. Braun Melsungen, Melsungen, Germany) after a 16 h fast and an acute medication. The glucose was administered via a feeding tube (15 g, 75 mm; Fine Science Tools, Heidelberg, Germany). The group relevant drugs will be given as shown below:

Group characterization and medication for the first and second OGTT

| | Animals | | |
|---|---|---|---|
| Group | (N) | Drug/Dose | Comments |
| CO | 5 | Placebo, 5 ml/kg b.w. | 0.5 mL/100 g b.w. 1% |
| | | | methylcellulose in saline at −5 min |
| GT | 5 | GT, 60 mg/kg b.w. | 60 mg GT solved in 5 mL 1% |
| | | | methylcellulose in saline, 0.5 mL/ |
| | | | 100 g b.w at −5 min |
| Rosiglitazone + GT | 5 | Rosiglitazone, 3 mg/kg | 3 mg Rosiglitazone solved in 2.5 ml |
| | | b.w. + P93/01, 60 mg/kg | 1% methylcellulose in saline, |
| | | b.w. | 0.250 ml/100 g b.w. at −30 min; |
| | | | 60 mg GT solved in 2.5 mL 1% |
| | | | methyosaline, 0.250 mL/100 g |
| | | | b.w at −5 min |
| Acarbose + GT | 5 | No Acarbose preload | 60 mg GT solved in 5 mL 1% |
| | | before OGTT; GT, 60 mg/kg | methylcellulose in saline, 0.5 mL/ |
| | | b.w. | 100 g b.w. at −5 min |
| Glibenclamide + GT | 5 | Glibenclamide, 5 mg/kg | 5 mg Glibenclamide solved in 2.5 ml |
| | | b.w. + GT, 60 mg/kg b.w. | 1% methylcellulose in saline, |
| | | | 0.250 mL/100 g b.w. at −40 min + 60 mg |
| | | | GT solved in 2.5 mL 1% |
| | | | methyl cellulose in saline, 0.250 mL/ |
| | | | 100 g b.w. at −5 min |
| Insulin + GT | 5 | Insulin, 2 IU SC + GT; 60 mg/kg | 2 IU Insulin SC Actrapid + 60 mg |
| | | b.w. | GT solved in 5 mL 1% |
| | | | methylcellulose in saline, 0.5 mL/ |
| | | | 100 g b.w. at −5 min. |

Blood samples were taken from tail veins to measure blood glucose and serum insulin at −15, ±0 min, 15, 30, 60, 90, 120 and 180 min (the latter time without insulin samples).

After first OGTT the animals in the groups was administered daily with the respective drugs at 08:00 AM and at 04:00 PM, respectively:

During the two weeks of medication morning blood glucose was measured before the 08:00 AM medication at Monday, Wednesday and Friday.

Food and water intake was determined every day during the time of medication.

All animals were weighted three times per week at 7:30 AM.

A second OGTT was performed after two weeks of medication (Day 15). The food will be withdrawn at 04:00 PM the day before (16 h fast). The OGTT was be performed with the pre-medication to defined times and oral glucose loading at ±0 min. Blood samples were taken from tail veins to measure blood glucose and serum insulin at −15, ±0 min, 15, 30, 60, 90, 120 and 180 min (the latter time without insulin samples).

After a wash-out period of one week a last OGTT was performed (>day 22). The food had been withdrawn at 04:00 PM the day before (16 h fast). The OGTT was performed with administration of placebo to all groups (this means 1% methylcellulose and SC saline, respectively) to the defined times and oral glucose loading at ±0 min. Blood samples were taken from tail veins to measure blood glucose and serum insulin at −15, ±0 min, 15, 30, 60, 90, 120 and 180 min (the latter time without insulin samples).

Glycated hemoglobin was measured before (Day −5) and after twice daily medication for >two weeks (Day 18).

Measurements:

Glucose-For determination of glucose 20 μL blood was collected at −15, ±0 min (before OGTT) and 15, 30, 60, 90, 120 and 180 min post OGTT.

Insulin-Insulin concentrations wer assayed by the antibody RIA method (Linco Research, Inc. St. Charles, Mo., USA).

Glycated hemoglobin-Percentage of glycated hemoglobin A (HbA1c) was estimated with the “DCA 2000R Hämoglobin A1c-Reagenz kit” (Bayer Vital GmbH, Fernwald, Germany).

Body weight-The body weight was measured using a platform balance (Scaltec, Heiligenstadt, Germany).

Mixed venous blood samples from the tails were collected into 20 μL glass capillaries, which will be placed in standard tubes filled with 1 ml solution for hemolysis (blood glucose measurement) and in sample tubes for serum insulin (50 μL blood). Blood samples in sample tubes were centrifuged immediately (12.000 rpm for 2 min) and serum for insulin analysis will be stored at −20° C. until analysis. Blood samples are labeled with Protocol Number, Date of sampling, Time of sampling, Animal Number and Type of sample.

Results:

First OGTT under medication was performed at the beginning of the study. Animals of all experimental groups showed no differences in their glucose tolerance after glucose load of 2 g/kg glucose. The same result was obtained regarding the baseline corrected glucose AUC_{1-180 min}. Baseline was set as glucose value at time t=0.

Blood glucose during OGTT-2 after 14 days subchronic medication:

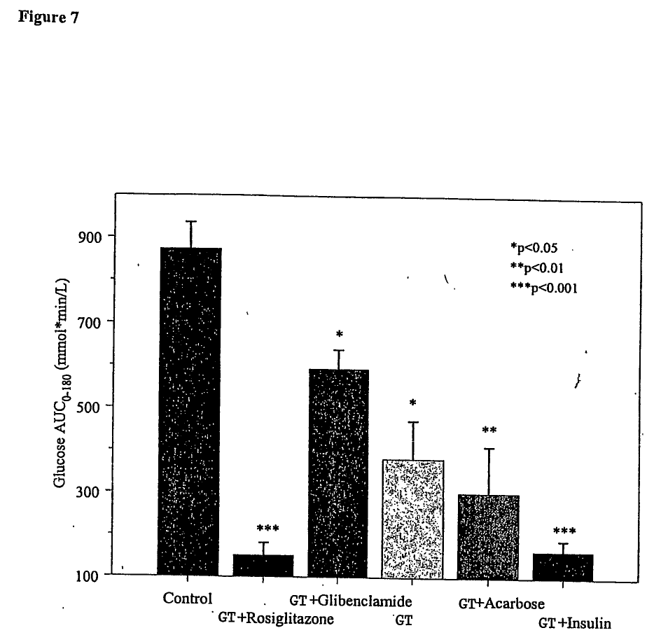

14 days subchronic treatment of fatty Zucker rats (fa/fa) has resulted in an improved glucose tolerance in all treatment groups versus Control. FIG. 7 shows the baseline corrected Area under the glucose-time-curve after glucose load of 2 g/kg glucose on day 14.

In conclusion subchronic administration of glutaminyl thiazolidine alone or in combination with Rosiglitazone, Glibenclamide, Acarbose or Insulin led to improved glucose tolerance.

1-26. (canceled)

27. A method for glycaemic control in a mammal, such as a human, which method comprises administering an effective amount of glutaminyl thiazolidine or glutaminyl pyrrolidine, or a pharmaceutically acceptable salt thereof, and another antidiabetic agent, to a mammal in need thereof.

28. A method for the treatment of diabetes mellitus and conditions associated with diabetes mellitus, the prediabetic state and/or obesity in a mammal, which method comprises administering an effective amount of glutaminyl thiazolidine or glutaminyl pyrrolidine, or a pharmaceutically acceptable salt thereof, and another antidiabetic agent, to a mammal in need thereof.

29. The use according to claim 27 or 28 for the treatment of Type 2 diabetes.

30. The according to claim 27 or 28 wherein the glutaminyl thiazolidine or glutaminyl pyrrolidine, or a pharmaceutically acceptable salt thereof, and the other antidiabetic agent are co-administered or administered sequentially or separately.

31. The use according to claim 27 or 28 wherein the glutaminyl thiazolidine or glutaminyl pyrrolidine, or a pharmaceutically acceptable salt thereof, and the other antidiabetic agent are administered orally.

32. The use according to claim 27 or 28 wherein the glutaminyl thiazolidine or glutaminyl pyrrolidine, or a pharmaceutically acceptable salt thereof, is glutaminyl thiazolidine hydrochloride.

33. The use according to claim 27 or 28 wherein the other antidiabetic agent is selected from an alpha glucosidase inhibitor, a biguanide, an insulin secretagogue or an insulin sensitiser.

34. The use according to claim 33, wherein the alpha glucosidase inhibitor is selected from acarbose, emiglitate, miglitol and voglibose.

35. The use according to claim 34, wherein the alpha glucosidase inhibitor is acarbose.

36. The use according to claim 33, wherein the biguanide is selected from metformin, buformin and phenformin.

37. The use according to claim 36, wherein the biguanide is metformin.

38. The use according to claim 33 wherein the insulin secretagogue is selected from glibenclamide, glipizide, gliclazide, glimepiride, tolazamide and tolbutamide, acetohexamide, carbutamide, chlorpropamide, glibomuride, gliquidone, glisentide, glisolamide, glisoxepide, glyclopyamide, glycylamide, glipentide repaglinide and nateglinide.

39. The use according to claim 33, wherein the insulin sensitiser is a PPARy agonist insulin sensitiser.

40. The use according to claim 33, wherein the insulin sensitiser is selected from troglitazone, ciglitazone, pioglitazone, englitazone and rosiglitazone.

41. A method for the treatment of diabetes mellitus and conditions associated with diabetes mellitus, the prediabetic state and/or obesity in a mammal, which method comprises administering an effective amount of glutaminyl thiazolidine hydrochloride and metformin, to a mammal in need thereof.

42. A pharmaceutical composition comprising glutaminyl thiazolidine or glutaminyl pyrrolidine, or a pharmaceutically acceptable salt thereof, and another antidiabetic agent, and a pharmaceutically acceptable carrier.

43. A pharmaceutical composition according to claim 42 wherein the glutaminyl thiazolidine or glutaminyl pyrrolidine, or a pharmaceutically acceptable salt thereof, is glutaminyl thiazolidine hydrochloride.

44. A pharmaceutical composition according to claim 42 or 43 wherein the other antidiabetic agent is as defined in claim 33.

45. A pharmaceutical composition comprising glutaminyl thiazolidine hydrochloride and metformin.
